(19) United States
(12) Patent Application Publication
(10) Pub. No.: US 2018/0325854 A1
(43) Pub. Date: Nov. 15, 2018

(54) USE OF PROTEASOME INHIBITORS TO TREAT OCULAR DISORDERS

(71) Applicant: ACCUITIS PHARMACEUTICALS, INC., Cumming, GA (US)

(72) Inventor: Richard COULON, Cumming, GA (US)

(21) Appl. No.: 15/773,920

(22) PCT Filed: Oct. 24, 2016
(86) PCT No.: PCT/US16/58402

§ 371 Date: May 4, 2018

Related U.S. Application Data
(60) Provisional application No. 62/251,937, filed on Nov. 6, 2015.

(51) Int. Cl. A61K 31/216; A61K 9/00; A61P 27/02

(52) U.S. Cl. CPC A61K 31/216; A61K 9/0048; A61K 45/06; A61K 2300/00; A61P 27/02

(57) ABSTRACT

Hydrocinnamate compounds that exhibit proteasome modulation activity, and in particular, proteasome inhibitory activity, can be used to topically or systemically treat ocular disorders associated with proteasome activity. The hydrocinnamate compounds can be applied to the eye, in any of a variety of ocular formulations, to treat ocular disorders, such as ocular rosacea, diabetic retinopathy, macular degeneration, and dry eye. The hydrocinnamate compounds can also be administered systemically to treat ocular disorders.

### FIELD OF THE INVENTION

The invention is generally in the area of treatment of ocular disorders, particularly ocular disorders mediated by proteasomes, and specifically including ocular inflammation, infections of the eye, and ocular disorders caused by inflammation and/or bacterial infections in the eye.

### BACKGROUND OF THE INVENTION

The ubiquitin-proteasome system (UPS) is the main intracellular pathway for modulated protein turnover, playing an important role in the maintenance of cellular homeostasis. It also exerts a protein quality control through degradation of oxidized, mutant, denatured, or misfolded proteins, and is involved in many biological processes where protein level regulation is necessary. This system allows the cell to modulate its protein expression pattern in response to changing physiological conditions and provides a critical protective role in health and disease.

Impairments of UPS function in the central nervous system (CNS) underlie a number of genetic and idiopathic diseases, many of which affect the retina. The relationship between UPS dysfunction is associated with numerous retina-specific illnesses with UPS involvement, such as retinitis pigmentosa, macular degenerations, glaucoma, diabetic retinopathy (DR), and aging-related impairments.

The ubiquitin-proteasome system (UPS) and autophagy are the two major intracellular protein degradation systems, and work collaboratively in many biological processes including development, apoptosis, aging, and countering oxidative injuries. In human retinal pigment epithelial cells (RPE) and ARPE-19 cells, proteasome inhibitors have been shown to increase the protein levels of autophagy-specific genes Atg5 and Atg7, and to enhance the conversion of microtubule-associated protein light chain (LC3) from LC3-I to its lipidative form, LC3-II.

Treatment with proteasome inhibitors can confer resistance to oxidative injury by a pathway involving inhibition of the PI3K-Akt-mTOR pathway and activation of autophagy. Proteasome inhibitors can also block development of posterior capsular opacification (PCO), and can also activate autophagy by inhibiting the PI3K-Akt-mTOR pathway as an anti-oxidation defense in human RPE cells.

TNF-alpha, IL-1beta, and TII induce expression of proinflammatory cytokines and ICAM-1 in hRPE cells through an NF-kappaB-dependent signal transduction pathway. This effect can be blocked by administration of a proteasome inhibitor, by preventing I kappaB degradation. Inhibition of NF-kappaB can also be a useful strategy to treat proliferative vitreoretinopathy and uveitis, ocular diseases initiated and perpetuated by cytokine activation, and is also constitutively active in human retinoblastoma cells and promotes their survival. This is described, for example, in Wang et al., “Suppression of NF-kappaB-dependent proinflammatory gene expression in human RPE cells by a proteasome inhibitor,” Invest Ophthalmol Vis Sci. 1999 February; 40(2):477-86.

Proteasome inhibitors can also induce apoptosis in human retinoblastoma cell lines, and as such, can also be used to treat retinoblastoma (see Invest Ophthalmol Vis Sci. 2007; 48(10):4706-19).

Proteasome inhibitors also provide protective effects in connection with ischemia-reperfusion injury in the retina. This effect is believed to be due to inhibition of the activation of NF-κB related to IR insult, and reducing the inflammatory signals and oxidative stress in the retina.

There are several other ocular disorders associated with inflammation, including ocular rosacea, dry eye, meibomian gland dysfunction/disease, posterior blepharitis, geographic atrophy, dry age related macular degeneration, wet age related macular degeneration, diabetic retinopathy, diabetic macular edema, uveitis, iritis, ocular injuries resulting from inflammation following eye surgeries, and inflammation caused by eye infections, whether by bacterial, viral, or other microbiological agents. Ocular injury is frequently associated with the inflammation caused by the immune response to the infection. Common eye infections include conjunctivitis (pink eye), blepharitis, trachoma and trichiasis, and these infections can affect any part of the eyes, from the eye lids to the cornea, and even the optic nerves in the back of the eye.

It would be advantageous to provide new compositions and methods for treating ocular disorders, including those associated with the ubiquitin-proteasome system (UPS), or with the production of pro-inflammatory cytokines. The present invention provides such compositions and methods.

### SUMMARY OF THE INVENTION

The present invention is based, in part, on the discovery that hydrocinnamate compounds that exhibit proteasome modulation activity, and in particular, proteasome inhibitory activity, can be used to topically or systemically treat ocular disorders associated with proteasome activity. For example, the hydrocinnamate compounds can be applied to the eye, in any of a variety of ocular formulations, to treat ocular disorders such as ocular rosacea. The hydrocinnamate compounds can also be administered systemically to treat ocular disorders.

Compositions and methods for treating or preventing ocular disorders associated with proteasome activity, and ocular disorders associated with an inflammatory response, including those caused by microbial infection, in both humans and non-human animals, are disclosed.

In one embodiment, the compositions include a cinnamate or dihydrocinnamate compound with proteasome inhibitor activity. In one aspect of this embodiment, the proteasome inhibitors compounds have one of the following formulas:

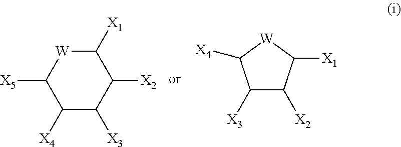

wherein W is selected from the group consisting of a methyl group, an alkyl group, a methylene group, an amine group, an acyl group, a carbonyl group, an oxygen atom, a sulfur atom, and wherein X₁ to X₅ are independently selected from the group consisting of a hydrogen atom, a halogen, a hydroxyl group, an ether group, an alkyl group, an aryl group, a nitro group, a cyano group, a thiol group, a thioether group, an amino group, an amido group, and an OR group, where R is an ester of a dihydrocinnamate; or

(ii) a dihydrocinnamate compound selected from the group consisting of

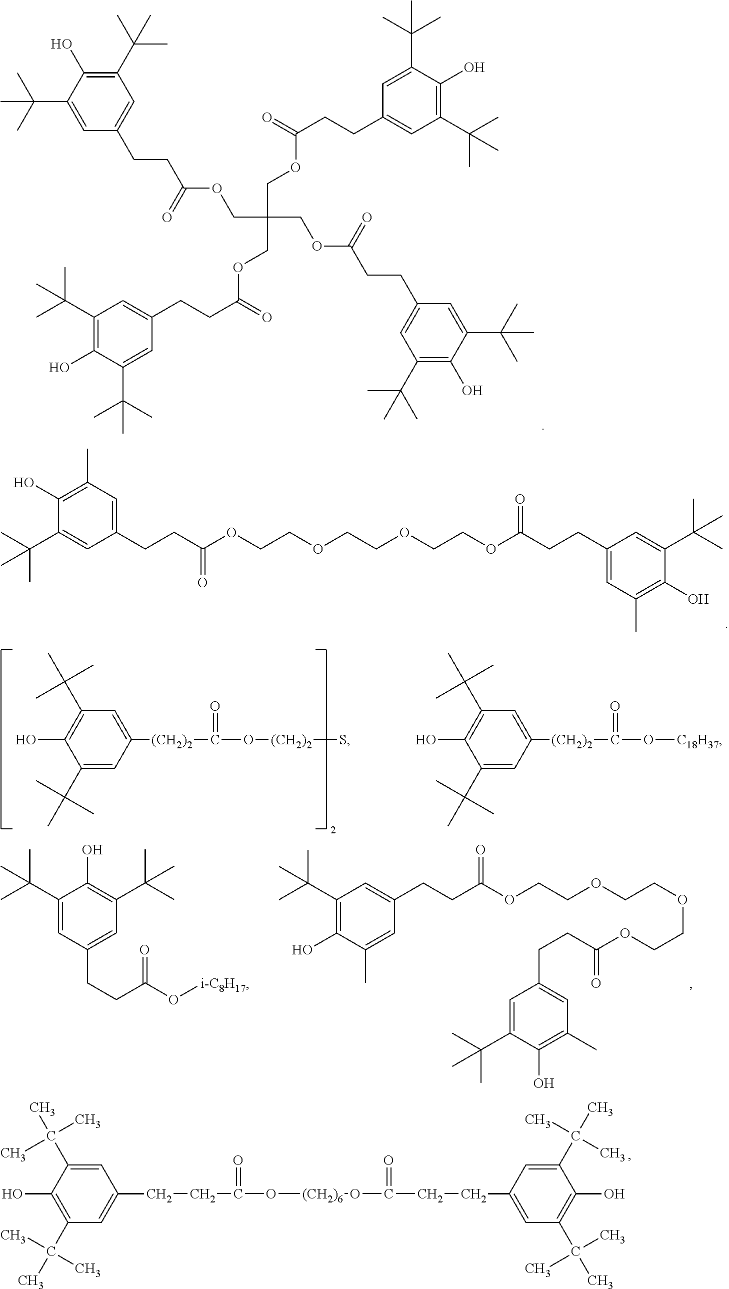

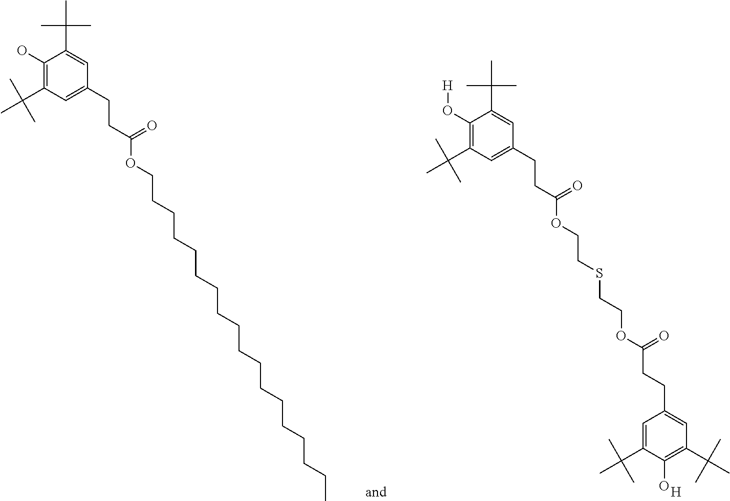

and analogs of the compounds in (i) or (ii) wherein one to three of the hydrogen atoms on the aromatic ring in the dihydrocinnamate moiety is replaced with a moiety selected from the group consisting of halogen, hydroxyl, ether, C₁₋₆ alkyl, C₆₋₁₀ aryl, nitro, cyano, thiol, thioester, amino, and amido. These compounds, and analogs thereof, are described, for example, in U.S. Pat. No. 8,809,283.

The compositions can further include appropriate carriers for optical administration, and the compositions can be used to treat or prevent the ocular disorders described herein.

Representative formulations include oral dosage forms, eye drops, gels, ointments, and other topically applied formulations, ocular inserts, formulations for injection, and formulations designed for iontophoretic administration. In some embodiments, the compositions are in the form of stabilized formulations (i.e., formulations which not require reconstitution with separately supplied sterile water), and in other embodiments, are in the form of formulations for reconstitution.

In one embodiment, oral dosage forms are used.

In one embodiment, the present invention further relates to stabilized aqueous proteasome inhibitor formulations. The stabilized formulations do not require reconstitution with separately supplied sterile water, aqueous solutions, or aqueous suspensions. Such stabilized formulations can be administered to the eye either prophylactically or to treat the disorders discussed herein.

In one embodiment, the ophthalmic formulations include water and proteasome inhibitor; and preferably have a pH in the range of between about 4.0 and about 7.0, more preferably from a pH of about 6.0 to about 7.5. The formulations can further include between about 0.4% and about 1.0% sodium chloride; between about 0.1% and about 2.0% citric acid; between about 0.1% and about 2.0% sodium citrate, between about 0.1% and about 10.0% proteasome inhibitor; and water.

The compositions can also include a lightly crosslinked carboxyl-containing polymer, which causes the solution to undergo a rapid increase in viscosity upon a pH rise associated with administration to tissues, such as those of the eye and the surrounding region.

A depot of proteasome inhibitor can be placed in contact with the eye for a sufficient length of time to allow a sufficient concentration of the proteasome inhibitor to diffuse into the cells of the targeted eye tissue(s). A therapeutically effective concentration of the proteasome inhibitor will remain in the tissue(s) for a considerable period. Accordingly, an advantage of certain preferred forms of the present invention is a simplified dosing regimen.

A proteasome inhibitor-containing depot can be formed by several means. One method of forming the depot involves including lightly crosslinked carboxyl containing polymers to the ophthalmic formulations, which causes the solutions to undergo a rapid increase in viscosity upon a pH rise associated with administration to tissues such as those of the eye and surrounding region.

A depot of the proteasome inhibitor can alternatively be formed by injecting a bolus of the composition into a target tissue. In one preferred method of ophthalmic administration the injection is intended to form a depot of material within the sclera, to accommodate extended release of the material to the surrounding tissues. Methods of intrascleral administration are discussed in U.S. Pat. No. 6,378,526 and U.S. Pat. No. 6,397,849.

Other means of forming a depot include the use of inserts loaded with a bolus of the drug to be delivered. Inserts placed under the eyelid have been used, for example, to deliver therapeutics to the ocular and periocular region.

In addition to the proteasome inhibitors described herein, the compositions can comprise one or more additional active agents. Representative additional active agents include, but are not limited to, anesthetics, anti-inflammatory agents, antimicrobial/anti-infective agents, anti-proliferative agents and combinations thereof.

The formulations described herein can be administered to the eye to treat disorders mediated by proteasomes, and/or which have an inflammatory component. The formulations are applied to an eye in an amount effective to treat or prevent the disorders. When administered with additional agents, the formulations can also provide anesthesia, prevent or treat inflammation, prevent unwanted cell proliferation and/or to provide treatment or prophylaxis of microbial infections.

Representative types of inflammatory ocular disorders that can be treated using the proteasome inhibitors described herein, for example, by topical application of compositions including one or more proteasome inhibitor, and also optionally including an anti-inflammatory agent, include ocular rosacea, wet and dry age-related macular degeneration (AMD), diabetic retinopathy (DR), glaucoma, neovascular glaucoma, retinal vasculitis, uveitis, such as posterior uveitis, keratoconjunctivitis sicca, conjunctivitis, retinitis secondary to glaucoma, neovascular glaucoma, episcleritis, scleritis, optic neuritis, retrobulbar neuritis, ocular inflammation following ocular surgery, ocular inflammation resulting from physical eye trauma, cataract, ocular allergy, dry eye, blepharitis, meibomian gland dysfunction, neurodegenerative disorders affecting the retina, including Alzheimer's, Parkinson's, and Huntington's diseases, and other retina-specific illnesses with UPS involvement, such as retinitis pigmentosa and age-related impairments.

Particularly where eye surgery is performed, prophylaxis can include prevention of post-surgical infection, and minimization of post-surgical inflammation. Representative types of eye surgeries for which the compositions can be used to provide anesthesia include laser eye surgery, refractive surgery, keratoplasty, keratotomy, keratomilleusis, cataract surgery, glaucoma surgery, canaloplasty, Karmra inlays, scleral reinforcement surgery, corneal surgery, vitreo-retinal surgery, retinal detachment repair, retinopexy, eye muscle surgery, surgery involving the lacrimal apparatus, insertion of implants into the eye, and eye removal.

Representative microbial infections that can be treated or prevented using combinations of the proteasome inhibitors and a suitable antimicrobial agent include viral, fungal, and bacterial infections in the eye, as well as ocular disorders resulting from these infections, such as trachoma, conjunctivitis, and the like. Representative bacteria that cause ocular infections in the inner or external eye include Haemophilus, Neisseria, Staphylococcus, Streptococcus, and Chlamydia.

Where an infection causes a disorder associated with an inflammatory component, the co-administration of anti-inflammatory agents and antimicrobials (i.e., antivirals, antibacterials, antifungals, antiparasitics, and the like), can be desirable. Other active agents, such as anti-proliferatives, anti-metabolites, VEGF inhibitors, prostaglandins, TGF-beta, mitomycin C, and antioxidants can also be added.

The present invention will be better understood with reference to the following detailed description.

### DETAILED DESCRIPTION

The invention described herein relates to compositions and methods for using proteasome inhibitors to treat ocular disorders, including those mediated by proteasomes, those associated with an inflammatory component, and those associated with infections, including viral, bacterial, fungal, and parasitic ocular infections.

The proteasome inhibitor can be administered alone or in combination with one or more additional active agents. Where the disorder is associated with an ocular infection, the additional active agent can be an antibiotic, and where the disorder is associated with inflammation, or the patient has eye surgery which can result in inflammation, the additional active agent can be an anti-inflammatory agent.

The present invention will be better understood with reference to the following detailed description, and with respect to the following definitions.

### Definitions

The term “an effective amount” refers to the amount of proteasome inhibitor, alone or in combination with one or more antibiotics, needed to eradicate the ocular infection, and/or, in combination with an anti-inflammatory agent, to eradicate the bacterial cause and inflammatory symptoms associated with various ocular disorders.

By “administering” is meant a method of giving one or more unit doses of an antibacterial pharmaceutical composition to an animal (e.g., topical, oral, intravenous, intraperitoneal, or intramuscular administration). The method of administration may vary depending on various factors, e.g., the components of the pharmaceutical composition, site of the potential or actual bacterial infection, bacteria involved, and severity of the actual bacterial infection.

By “bacteria” is meant a unicellular prokaryotic microorganism that usually multiplies by cell division.

By “ocular bacterial infection” is meant the invasion of an eye in a host animal by pathogenic bacteria. For example, the infection may include the excessive growth of bacteria that are normally present in or on the body of an animal or growth of bacteria that are not normally present in or on the animal. More generally, a bacterial infection can be any situation in which the presence of a bacterial population(s) is damaging to a host animal. Thus, an animal is “suffering” from an ocular bacterial infection when an excessive amount of a bacterial population is present in or on the animal's eye, or when the presence of a bacterial population(s) is damaging the cells or other tissue in the eye of the animal.

By “persistent bacterial infection” is meant an infection that is not completely eradicated through standard treatment regimens using antibiotics. Persistent bacterial infections are caused by bacteria capable of establishing a cryptic phase or other non-multiplying form of a bacterium and may be classified as such by culturing bacteria from a patient and demonstrating bacterial survival in vitro in the presence of antibiotics or by determination of anti-bacterial treatment failure in a patient. Trachoma is an example of a persistent ocular bacterial infection.

As used herein, a persistent infection in a patient includes any recurrence of an infection, after receiving antibiotic treatment, from the same species more than two times over the period of two or more years or the detection of the cryptic phase of the infection in the patient. An in vivo persistent infection can be identified through the use of a reverse transcriptase polymerase chain reaction (RT-PCR) to demonstrate the presence of 16S rRNA transcripts in bacterially infected cells after treatment with one or more antibiotics (Antimicrob. Agents Chemother. 12:3288-3297, 2000).

Ocular viral infections include common pink eye, ocular herpes, which occurs with exposure to the Herpes simplex virus, shingles, and Ebola, which persists in the eye.

Ocular fungal infections include fungal keratitis, which is often associated with Fusarium fungi.

Acanthamoeba keratitis and river blindness are examples of parasitic ocular infections.

By “chronic disease” is meant a disease that is inveterate, of long continuance, or progresses slowly, in contrast to an acute disease, which rapidly terminates. A chronic disease may begin with a rapid onset or in a slow, insidious manner but it tends to persist for several weeks, months or years, and has a vague and indefinite termination.

By “immunocompromised” is meant a person who exhibits an attenuated or reduced ability to mount a normal cellular or humoral defense to challenge by infectious agents, e.g., viruses, bacterial, fungi, and protozoa. Persons considered immunocompromised include malnourished patients, patients undergoing surgery and bone narrow transplants, patients undergoing chemotherapy or radiotherapy, neutropenic patients, HIV-infected patients, trauma patients, burn patients, patients with chronic or resistant infections such as those resulting from myelodysplastic syndrome, and the elderly, all of who may have weakened immune systems.

By “inflammatory disease” is meant a disease state characterized by (1) alterations in vascular caliber that lead to an increase in blood flow, (2) structural changes in the microvasculature that permit the plasma proteins and leukocytes to leave the circulation, and (3) emigration of the leukocytes from the microcirculation and their accumulation in the focus of injury. The classic signs of acute inflammation are erythema, edema, tenderness (hyperalgesia), and pain. Chronic inflammatory diseases are characterized by infiltration with mononuclear cells (e.g., macrophages, lymphocytes, and plasma cells), tissue destruction, and fibrosis. Non-limiting examples of inflammatory ocular diseases include ocular rosacea, trachoma, wet and dry age-related macular degeneration (AMD), diabetic retinopathy (DR), glaucoma, neovascular glaucoma, retinal vasculitis, uveitis, such as posterior uveitis, conjunctivitis, retinitis secondary to glaucoma, episcleritis, scleritis, optic neuritis, retrobulbar neuritis, ocular inflammation following ocular surgery, ocular inflammation resulting from physical eye trauma, cataract, ocular allergy and dry eye.

By “treating” is meant administering a pharmaceutical composition for prophylactic and/or therapeutic purposes. To “prevent disease” refers to prophylactic treatment of a patient who is not yet ill, but who is susceptible to, or otherwise at risk of, a particular disease. To “treat disease” or use for “therapeutic treatment” refers to administering treatment to a patient already suffering from a disease to improve the patient's condition. Thus, in the claims and embodiments, treating is the administration to a mammal either for therapeutic or prophylactic purposes.

The term “pharmaceutically acceptable salt” is used throughout the specification to describe any pharmaceutically acceptable salt form of the proteasome inhibitors described herein. Pharmaceutically acceptable salts include those derived from pharmaceutically acceptable inorganic or organic bases and acids. Citric acid is a specific example of a suitable acid. Suitable salts include those derived from alkali metals such as potassium and sodium, alkaline earth metals such as calcium and magnesium, among numerous other acids well known in the pharmaceutical art.

Pharmaceutically acceptable salts include also include complexes with amines, including ammonia, primary, secondary and tertiary amines. The amines can form salts or partial salts with one or more of the phenolic hydrogens.

The present invention satisfies an existing need for compounds that effective in treating ocular disorders mediated by proteasomes, or associated with inflammation.

### I. Proteasome Inhibitors

The compositions include a proteasome inhibitor of the formulas:

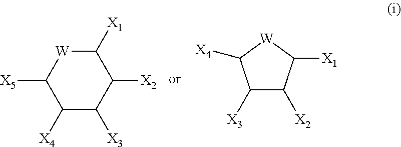

wherein W is selected from the group consisting of a methyl group, an alkyl group, a methylene group, an amine group, an acyl group, a carbonyl group, an oxygen atom, a sulfur atom, and wherein X₁ to X₅ are independently selected from the group consisting of a hydrogen atom, a halogen, a hydroxyl group, an ether group, an alkyl group, an aryl group, a nitro group, a cyano group, a thiol group, a thioether group, an amino group, an amido group, and an OR group, where R is an ester of a dihydrocinnamate; or

(ii) a dihydrocinnamate compound selected from the group consisting of

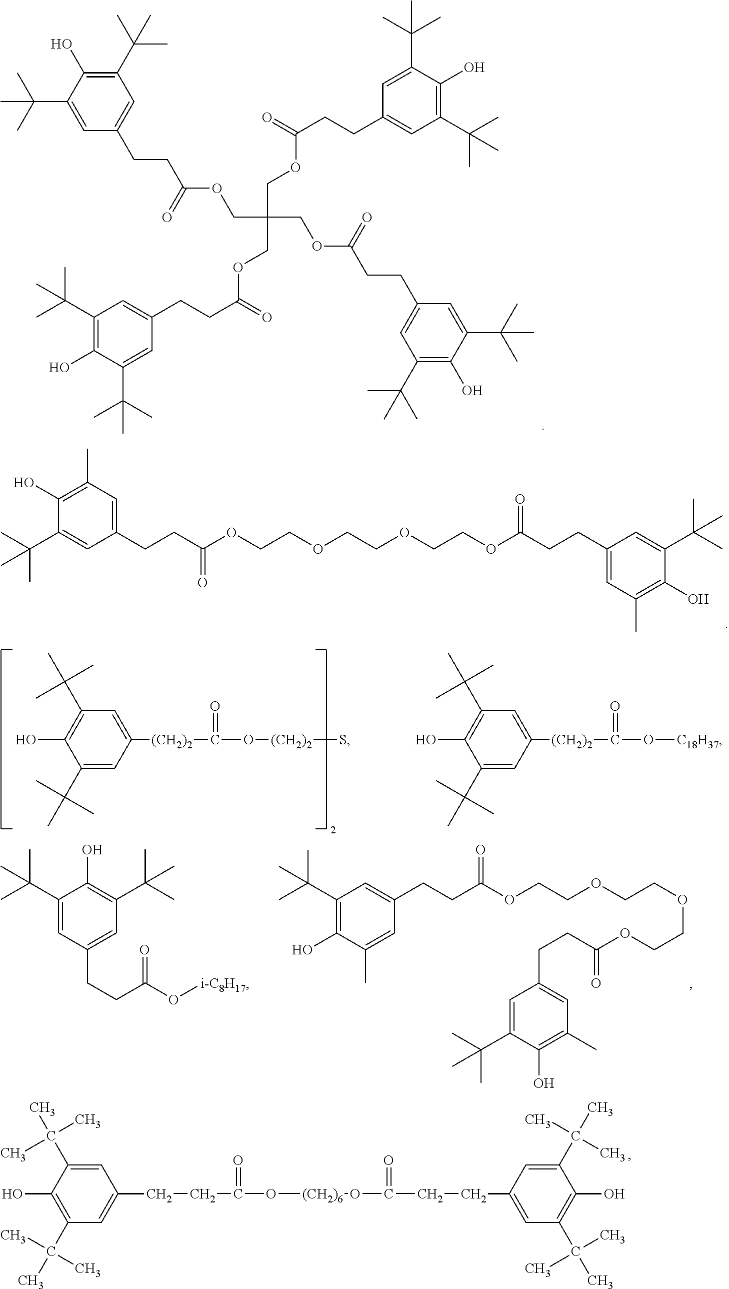

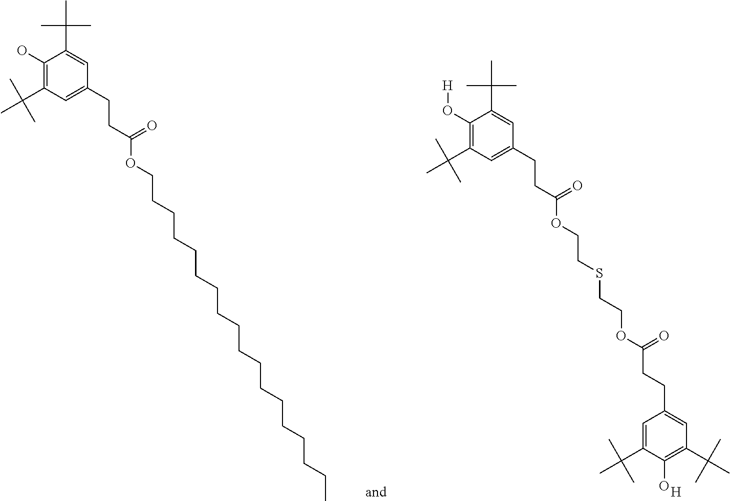

and analogs of the compounds in (i) or (ii) wherein one to three of the hydrogen atoms on the aromatic ring in the dihydrocinnamate moiety is replaced with a moiety selected from the group consisting of halogen, hydroxyl, ether, C₁₋₆ alkyl, C₆₋₁₀ aryl, nitro, cyano, thiol, thioester, amino, and amido. These compounds, and analogs thereof, are described, for example, in U.S. Pat. No. 8,809,283.

Analogs of the compounds discussed above, wherein the compounds have multiple cinnamate or hydrocinnamate esters, include those wherein one or more of the esters is hydrolyzed to the free OH group (i.e., partial esters of PTTC and other proteasome inhibitors).

Complexes of the compounds described herein with albumin, such as human serum albumin (HSA), are also within the scope of the invention. Looking at the amino acid sequence of HSA, there are a number of ionizable groups: 116 total acidic groups (98 carboxyl and 18 phenolic OH) and 100 total basic groups (60 amino, 16 imidazolyl, and 24 guanidyl). These complexes can be formed by mixing the compounds with albumin, or by complexing albumin with the compounds described herein using a multivalent cation. Multivalent cations can, for example, can bridge phenolic groups on the compounds described herein, and phenolic groups on HSA.

Analogs also include hydrocinnamate and cinnamate esters of polyhydric alcohols like pentaerythritol, for example, pentaerythritol esters with 3,2, and 1 acyl groups. As used herein, an acyl group is an ester group with a C₁₋₂₀ alkyl, C₂₋₂₀ alkenyl, ₂₋₂₀alkynyl, or C₆ or C₁₀ aryl moiety.

The compounds described herein all include at least one aryl ring, and each ring can, independently, be further substituted with one or more substituents, as defined herein. Those skilled in the art will readily understand that incorporation of other substituents onto an aryl ring used as a starting material to prepare the compounds described herein, and other positions in the compound framework, can be readily realized. Such substituents can provide useful properties in and of themselves or serve as a handle for further synthetic elaboration.

Benzene rings can be substituted using known chemistry, including the reactions discussed below. For example, alkyl substituents can be added using friedel craft alkylation reactions. Biphenyl compounds can be synthesized by treating aryl phenylmagnesium bromides with copper salts, by the oxidative dehydrogenation of the aryl rings, or the dealkylation of toluene or other methyl-substituted aromatic rings.

Aryl rings can be nitrated, and the resulting nitro group on the aryl ring reacted with sodium nitrite to form a diazonium salt. The diazonium salt can be manipulated using known chemistry to form various substituents on a benzene ring.

Diazonium salts can be halogenated using various known procedures, which vary depending on the particular halogen. Examples of suitable reagents include bromine/water in concentrated HBr, thionyl chloride, pyr-ICl, fluorine and Amberlyst-A.

A number of other analogs, bearing substituents in the diazotized position, can be synthesized from the corresponding amino compounds, via the diazo intermediate. The diazo compounds can be prepared using known chemistry, for example, as described above.

Nitro derivatives can be reduced to the amine compound by reaction with a nitrite salt, typically in the presence of an acid. Other substituted analogs can be produced from diazonium salt intermediates, including, but are not limited to, hydroxy, alkoxy, fluoro, chloro, iodo, cyano, and mercapto, using general techniques known to those of skill in the art.

For example, hydroxy-aromatic analogues can be prepared by reacting the diazonium salt intermediate with water. Halogens on an aryl ring can be converted to Grignard or organolithium reagents, which in turn can be reacted with a suitable aldehyde or ketone to form alcohol-containing side chains. Likewise, alkoxy analogues can be made by reacting the diazo compounds with alcohols. The diazo compounds can also be used to synthesize cyano or halo compounds, as will be known to those skilled in the art. Mercapto substitutions can be obtained using techniques described in Hoffman et al., J. Med. Chem. 36: 953 (1993). The mercaptan so generated can, in turn, be converted to an alkylthio substitutent by reaction with sodium hydride and an appropriate alkyl bromide. Subsequent oxidation would then provide a sulfone. Acylamido analogs of the aforementioned compounds can be prepared by reacting the corresponding amino compounds with an appropriate acid anhydride or acid chloride using techniques known to those skilled in the art of organic synthesis.

Hydroxy-substituted analogs can be used to prepare corresponding alkanoyloxy-substituted compounds by reaction with the appropriate acid, acid chloride, or acid anhydride. Likewise, the hydroxy compounds are precursors of both the aryloxy via nucleophilic aromatic substitution at electron deficient aromatic rings. Such chemistry is well known to those skilled in the art of organic synthesis. Ether derivatives can also be prepared from the hydroxy compounds by alkylation with alkyl halides and a suitable base or via Mitsunobu chemistry, in which a trialkyl- or triarylphosphine and diethyl azodicarboxylate are typically used. See Hughes, Org. React. (N.Y.) 42: 335 (1992) and Hughes, Org. Prep. Proced. Int. 28: 127 (1996) for typical Mitsunobu conditions.

Cyano-substituted analogs can be hydrolyzed to afford the corresponding carboxamido-substituted compounds. Further hydrolysis results in formation of the corresponding carboxylic acid-substituted analogs. Reduction of the cyano-sub stituted analogs with lithium aluminum hydride yields the corresponding aminomethyl analogs. Acyl-substituted analogs can be prepared from corresponding carboxylic acid-substituted analogs by reaction with an appropriate alkyllithium using techniques known to those skilled in the art of organic synthesis.

Carboxylic acid-substituted analogs can be converted to the corresponding esters by reaction with an appropriate alcohol and acid catalyst. Compounds with an ester group can be reduced with sodium borohydride or lithium aluminum hydride to produce the corresponding hydroxymethyl-substituted analogs. These analogs in turn can be converted to compounds bearing an ether moiety by reaction with sodium hydride and an appropriate alkyl halide, using conventional techniques. Alternatively, the hydroxymethyl-substituted analogs can be reacted with tosyl chloride to provide the corresponding tosyloxymethyl analogs, which can be converted to the corresponding alkylaminoacyl analogs by sequential treatment with thionyl chloride and an appropriate alkylamine. Certain of these amides are known to readily undergo nucleophilic acyl substitution to produce ketones.

Hydroxy-substituted analogs can be used to prepare N-alkyl- or N-arylcarbamoyloxy-substituted compounds by reaction with N-alkyl- or N-arylisocyanates. Amino-substituted analogs can be used to prepare alkoxycarboxamido-substituted compounds and urea derivatives by reaction with alkyl chloroformate esters and N-alkyl- or N-arylisocyanates, respectively, using techniques known to those skilled in the art of organic synthesis.

Any of the aforementioned substituents can be present on any or all of the aromatic rings in the compounds described herein.

### II. Pharmaceutical Compositions/Formulations

The pharmaceutical compositions and formulations described herein include proteasome inhibitor as described herein, a suitable carrier, and, optionally, one or more other active agents.

Proteasome Inhibitor Formulations

The proteasome inhibitor used in the invention described herein can be in any suitable form that provides suitable bioavailability. Drug delivery devices and formulations that locally deliver proteasome inhibitor to the eye are described in detail herein. However, in some embodiments, the disorders can be treated with an oral dosage form of the proteasome inhibitor.

Stabilized aqueous formulations comprising the proteasome inhibitors described herein can be prepared under strictly controlled Good Manufacturing Practice (GMP) conditions, ensuring both the quality and uniformity of the materials while avoiding the requirement for reconstitution by the pharmacist, physician, or patient. Moreover, sufficiently stable formulations are amendable to commercial transportation and can dispensed and administered without concern that the active component will be unacceptably degraded.

In addition, suitably stable formulations can be dispensed for administration over an extended course of treatment, or packaged in single dose forms suitable for direct administration by a patient or physician without the effort or concern over reconstitution. Stable aqueous formulations of the proteasome inhibitor can be administered topically.

The aqueous compositions (solutions or suspensions) preferably use water that has no physiologically or ophthalmically harmful constituents. Typically purified or deionized water is used. The pH is adjusted by adding any physiologically and ophthalmically acceptable pH adjusting acids, bases, or buffers to within the range of about 5.0 to about 7.0, more preferably from about 5.8 to about 6.8, more preferably about 6.0 to about 6.5, more preferably at a pH of about 6.2 to about 6.4, more preferably about 6.25 to about 6.35, or more preferably about 6.3. In alternative embodiments, the proteasome inhibitor compositions of the present invention can be adjusted to a pH in the range of 5.0 to about 6.0, or more preferably about 5.5 to about 5.95, or more preferably 5.6 to 5.9. Any of the aforementioned ranges can be used with any of the compositions of the present invention, including, without limitation, intravenous and topical embodiments. Examples of acids include acetic, boric, citric, lactic, phosphoric, hydrochloric, and the like, and examples of bases include potassium hydroxide, sodium hydroxide, sodium phosphate, sodium borate, sodium citrate, sodium acetate, sodium lactate, tromethamine, THAM (tris-hydroxymethylamino-methane), and the like. Salts and buffers include but are not limited to citrate/dextrose, sodium bicarbonate, ammonium chloride and mixtures of the aforementioned acids and bases. The pH is preferably adjusted by adding sodium hydroxide.

In preferred embodiments of this invention, wherein the composition is intended for topical administration to ocular or periocular tissues, the composition may be formulated for application as a liquid drop, ointment, a viscous solution or gel, a ribbon, or a solid. The composition can be topically applied, for example, without limitation, to the front of the eye, under the upper eyelid, on the lower eyelid and in the cul-de-sac.

In an alternative embodiment the stabilized formulation of proteasome inhibitor is formulated as a solid, semi-solid, powdered, or lyophilized composition, which upon addition of water or aqueous solutions produces a stabilized proteasome inhibitor formulation having a pH of about 4.0 to about 7.0, more preferably of about 5.8 to about 6.8, more preferably from about 6.0 to about 6.6, more preferably of about 6.2 to about 6.4, more preferably of about 6.25 to 6.35, and even more preferably about 6.3.

Representative formulations are described in detail below.

Ocular Formulations

Current methods for ocular delivery include topical administration (eye drops or other suitable topical formulations for direct administration to the eye), subconjunctival injections, periocular injections, intravitreal injections, surgical implants, and systemic routes. Any of these routes can be used, as appropriate, depending on the particular disorder to be treated.

Intravitreal injections, periocular injections, and sustained-release implants can be used to achieve therapeutic levels of drugs in ocular tissues. Eye drops are useful in treating conditions affecting either the exterior surface of the eye or tissues in the front of the eye, and some formulations can penetrate to the back of the eye for treatment of retinal diseases.

Certain disorders affect tissues at the back of the eye, where treatment is difficult to deliver. In these embodiments, iontophoresis can be used to deliver the compounds described herein to the back of the eye. For example, the ocular iontophoresis system, OcuPhor™, can deliver drugs safely and non-invasively to the back of the eye (Iomed). Iontophoresis uses a small electrical current to transport ionized drugs into and through body tissues. Care must be taken not to use too high of a current density, which can damage eye tissues.

Iontophoresis typically involves using a drug applicator, a dispersive electrode, and an electronic iontophoresis dose controller. The drug applicator can be a small silicone shell that contains a conductive element, such as silver-silver chloride. A hydrogel pad can absorb the drug formulation. A small, flexible wire can connect the conductive element to the dose controller. The drug pad can be hydrated with a drug solution immediately before use, with the applicator is placed on the sclera of the eye under the lower eyelid. The eyelid holds the applicator in place during treatment. The drug dose and rate of administration can be controlled by programming and setting the electronic controller.

Solid/Semi-Solid/Powdered/Lyophilized Compositions

Solid, semi-solid, powdered, or lyophilized composition may be prepared and packaged for single dose or multiple dose delivery. The solid, semi-solid, powdered, or lyophilized compositions may also contain one or more additional medicaments or pharmaceutically acceptable excipients compatible with the intended route of administration. In a preferred embodiment for ocular administration, the solid, semi-solid, powdered, or lyophilized compositions may also contain polymeric suspending agents. The reconstitutable formulations of stabilized proteasome inhibitor thus provide for compositions having the advantages of a shelf life comparable to that of, and additionally, the extended shelf life of the stablized aqueous formulations described herein.

Formulations for Topical Administration

The proteasome inhibitor compositions suitable for topical administration to the eye or periocular tissue can include one or more “ophthalmically acceptable carriers.” Such carriers are well known to those of skill in the art.

Although proteasome inhibitor can reach many of the tissues and fluids of the eye by oral administration, the proteasome inhibitors described herein are amenable to topical administration to eye and periocular tissues. The proteasome inhibitor can be supplied to the eye surface in a variety of ways, including as an aqueous ophthalmic solution or suspension, as an ophthalmic ointment, and as an ocular insert, but application is not limited thereto. Any technique and ocular dosage form that supplies proteasome inhibitor to the external eye surface is included within the definition of “topically applying.” Although the external surface of the eye is typically the outer layer of the conjunctiva, it is possible that the sclera, cornea, or other ocular tissue could be exposed such as by rotation of the eye or by surgical procedure, and thus be an external surface. For the purposes of this application, periocular tissues are defined as those tissues in contact with the lachrymal secretions, including the inner surface of the eye lid, the tissues of the orbit surrounding the eye, and the tissues and ducts of the lachrymal gland.

The amount of proteasome inhibitor topically supplied is effective to treat or prevent a disorder mediated by proteasomes, or associated with inflammation, in a tissue of the eye. More specifically, the concentration within the ocular tissue is desired to be at least about 0.25 μg/g, preferably at least about 1 μg/g, and more preferably at least about 10 μg/g. The amount of proteasome inhibitor actually supplied to the external eye surface will almost always be higher than the tissue concentration. This reflects the penetration hold up of the proteasome inhibitor by the outer tissue layers of the eye and that penetration is, to some extent, concentration driven. Thus, supplying greater amounts to the exterior will drive more of the proteasome inhibitor into the tissues. Delivery of formulations as a depot will advantageously maintain the concentration of the proteasome inhibitor in the affected tissues for a period of at least about 2 hours, or more preferably at least about 4 hours, more preferably at least about 8 hours, or more preferably at least about 12 hours.

Where a series of applications are typically employed in a topical administration dosing regimen, it is possible that one or more of the earlier applications will not achieve an effective concentration in the ocular tissue, but that a later application in the regimen will achieve an effective concentration. This is contemplated as being within the scope of topically applying proteasome inhibitor in an effective amount. However, generally a single application, such as consisting of one or two drops, provides a therapeutically effective concentration of the proteasome inhibitor within a tissue of the eye. Indeed, although dependent on the amount and form of the ophthalmic composition, a single application will typically provide a therapeutically effective amount of the proteasome inhibitor within a tissue of the eye for at least about 2, more preferably about 4, more preferably about 8, more preferably about 12, and more preferably at least about 18 hours. As discussed above, the stabilized proteasome inhibitor compositions of this invention may be topically administered to a variety of tissues, including the eye, to provide prophylaxis or treatment of ocular disorders mediated by proteasomes.

Intrascleral Injection

In one embodiment, proteasome inhibitor is administered to eye tissues by intrascleral injection, as disclosed in U.S. Pat. Nos. 6,397,849 and 6,378,526. Administration by means of intrascleral injection can advantageously be employed to provide the proteasome inhibitors described herein to the tissues of the posterior segment of the eye. Depending on the injection conditions, the proteasome inhibitor will (1) form a depot within the scleral layer and diffuse into the underlying tissue layers such as the choroid and/or retina, (2) be propelled through the scleral layer and into the underlying layers, or (3) a combination of both (1) and (2).

Formation of a Depot of Proteasome Inhibitor in the Eye of a Patient

A preferred form of the present invention for topical ophthalmic administration provides for achieving a sufficiently high tissue concentration of proteasome inhibitor with a minimum of doses so that a simple dosing regimen can be used to treat or prevent the ocular disorders described herein. To this end, a preferred technique involves forming or supplying a depot of proteasome inhibitor in contact with the external surface of the eye. A depot refers to a source of proteasome inhibitor that is not rapidly removed by tears or other eye clearance mechanisms. This allows for continued, sustained high concentrations of proteasome inhibitor to be present in the fluid on the external surface of the eye by a single application. In general, it is believed that absorption and penetration are dependent on both the dissolved drug concentration and the contact duration of the external tissue with the drug-containing fluid. As the drug is removed by clearance of the ocular fluid and/or absorption into the eye tissue, more drug is provided, e.g. dissolved, into the replenished ocular fluid from the depot.

Accordingly, the use of a depot more easily facilitates loading of the ocular tissue in view of the typically slow and low penetration rate of the generally water-insoluble or poorly soluble proteasome inhibitor. The depot, which retains a bolus of concentrated drug, can effectively slowly “pump” the proteasome inhibitor into the ocular tissue. As the proteasome inhibitor penetrates the ocular tissue, it is accumulated therein and not readily removed due to its long half-life. As more proteasome inhibitor is “pumped” in, the tissue concentration increases and the minimum inhibitory concentration threshold is eventually reached or exceeded, thereby loading the ocular tissue with the proteasome inhibitor. Thus, depending on the depot, one or two applications may provide a complete dosing regimen. In one embodiment, the dosing regimen involves one to two doses per day over a one to three day period, more preferably one or two doses in a single day, to provide in vivo at least a 6 day treatment and more typically a 6 to 14 day treatment.

A depot can take a variety of forms so long as the proteasome inhibitor can be provided in sufficient concentration levels therein and is releasable therefrom, and that the depot is not readily removed from the eye. A depot generally remains for at least about 30 minutes after administration, preferably at least 2 hours, and more preferably at least 4 hours. The term “remains” means that neither the depot composition nor the proteasome inhibitor is exhausted or cleared from the surface of the eye prior to the indicated time. In some embodiments, the depot can remain for up to eight hours or more. Typical ophthalmic depot forms include aqueous polymeric suspensions, ointments, and solid inserts. Polymeric suspensions are the most preferred form for the present invention and will be discussed subsequently.

Ointments

Ointments, which are essentially an oil-based delivery vehicle, are a well known compositions for topical administration. Common bases for the preparation of ointments include mineral oil, petrolatum and combinations thereof, but oil bases are not limited thereto. When used for ophthalmic administration, ointments are usually applied as a ribbon onto the lower eyelid. The disadvantage of ointments is that they can be difficult to administer, can be messy, and can be uncomfortable or inconvenient to the patient. Moreover, temporarily blurred vision is a common difficulty encountered when they are employed for ophthalmic administration.

Inserts

Inserts are another well-known ophthalmic dosage form and comprise a matrix containing the active ingredient. The matrix is typically a polymer, and the active ingredient is generally dispersed therein or bonded to the polymer matrix. The active ingredient is slowly released from the matrix through dissolution or hydrolysis of the covalent bond, etc. In some embodiments, the polymer is bioerodible (soluble) and the dissolution rate thereof can control the release rate of the active ingredient dispersed therein. In another form, the polymer matrix is a biodegradable polymer that breaks down, such as by hydrolysis, to thereby release the active ingredient bonded thereto or dispersed therein. The matrix and active ingredient can be surrounded with a polymeric coating, such as in the sandwich structure of matrix/matrix+active/matrix, to further control release, as is well known in the art. The kinds of polymers suitable for use as a matrix are well known in the art. The proteasome inhibitor can be dispersed into the matrix material or dispersed amongst the monomer composition used to make the matrix material prior to polymerization. The amount of proteasome inhibitor is generally from about 0.1 to 50%, more typically about 2 to 20%. The insert can be placed, depending on the location and the mechanism used to hold the insert in position, by either the patient or the doctor, and is generally located under the upper eye lid. A variety of shapes and anchoring configurations are recognized in the art. Preferably a biodegradable or bioerodible polymer matrix is used so that the spent insert does not have to be removed. As the biodegradable or bioerodible polymer is degraded or dissolved, the trapped proteasome inhibitor is released. Although inserts can provide long term release and hence only a single application of the insert may be necessary, they are generally difficult to insert and are uncomfortable to the patient.

Aqueous Polymeric Suspensions

A preferred form of the stabilized proteasome inhibitor composition for administration of proteasome inhibitor to the ocular and periocular tissues is an aqueous polymeric suspension. Here, at least one of the proteasome inhibitor or the polymeric suspending agent is suspended in an aqueous medium having the properties as described above. The proteasome inhibitor may be in suspension, although in the preferred pH ranges the proteasome inhibitor may also be in solution (water soluble), or both in solution and in suspension. It is possible for significant amounts of the proteasome inhibitor to be present in suspension. The polymeric suspending agent is preferably in suspension (i.e. water insoluble and/or water swellable), although water soluble suspending agents are also suitable for use with a suspension of the proteasome inhibitor antibiotic. The suspending agent serves to provide stability to the suspension and to increase the residence time of the dosage form on the eye. It can also enhance the sustained release of the drug in terms of both longer release times and a more uniform release curve.

Examples of polymeric suspending agents include dextrans, polyethylene glycols, polyvinylpyrolidone, polysaccharide gels, Gelrite®, cellulosic polymers like hydroxypropyl methylcellulose, and carboxy-containing polymers such as polymers or copolymers of acrylic acid, as well as other polymeric demulcents. A preferred polymeric suspending agent is a water swellable, water insoluble polymer, especially a crosslinked carboxy-containing polymer.

Crosslinked carboxy-containing polymers used in practicing this invention are, in general, well known in the art. In a preferred embodiment such polymers may be prepared from at least about 90%, and preferably from about 95% to about 99.9% by weight, based on the total weight of monomers present, of one or more carboxy-containing monoethylenically unsaturated monomers (also occasionally referred to herein as carboxy-vinyl polymers). Acrylic acid is the preferred carboxy-containing monoethylenically unsaturated monomer, but other unsaturated, polymerizable carboxy-containing monomers, such as methacrylic acid, ethacrylic acid, .beta.-methylacrylic acid (crotonic acid), cis-alpha-methylcrotonic acid (angelic acid), trans-alpha-methylcrotonic acid (tiglic acid), alpha-butylcrotonic acid, alpha-phenylacrylic acid, alpha-benzylacrylic acid, alpha-cyclohexylacrylic acid, .beta.-phenylacrylic acid (cinnamic acid), coumaric acid (o-hydroxycinnamic acid), umbellic acid (p-hydroxycoumaric acid), and the like can be used in addition to or instead of acrylic acid.

Such polymers may be crosslinked by a polyfunctional crosslinking agent, preferably a difunctional crosslinking agent. The amount of crosslinking should be sufficient to form insoluble polymer particles, but not so great as to unduly interfere with sustained release of the proteasome inhibitor antibiotic. Typically the polymers are only lightly crosslinked. Preferably the crosslinking agent is contained in an amount of from about 0.01% to about 5%, preferably from about 0.1% to about 5.0%, and more preferably from about 0.2% to about 1%, based on the total weight of monomers present. Included among such crosslinking agents are non-polyalkenyl polyether difunctional crosslinking monomers such as divinyl glycol; 2,3-dihydroxyhexa-1,5-diene; 2,5-dimethyl-1,5-hexadiene; divinylbenzene; N,N-diallylacrylamide; N,N-diallymethacrylamide and the like. Also included are polyalkenyl polyether crosslinking agents containing two or more alkenyl ether groupings per molecule, preferably alkenyl ether groupings containing terminal H₂C═C<groups, prepared by etherifying a polyhydric alcohol containing at least four carbon atoms and at least three hydroxyl groups with an alkenyl halide such as allyl bromide or the like, e.g., polyallyl sucrose, polyallyl pentaerythritol, or the like; see, e.g., Brown U.S. Pat. No. 2,798,053, the entire contents of which are incorporated herein by reference. Diolefinic non-hydrophilic macromeric crosslinking agents having molecular weights of from about 400 to about 8,000, such as insoluble di- and polyacrylates and methacrylates of diols and polyols, diisocyanate-hydroxyalkyl acrylate or methacrylate reaction products of isocyanate terminated prepolymers derived from polyester diols, polyether diols or polysiloxane diols with hydroxyalkylmethacrylates, and the like, can also be used as the crosslinking agents; see, e.g., Mueller et al. U.S. Pat. Nos. 4,192,827 and 4,136,250, the entire contents of each patent being incorporated herein by reference.

The crosslinked carboxy-vinyl polymers may be made from a carboxy-vinyl monomer or monomers as the sole monoethylenically unsaturated monomer present, together with a crosslinking agent or agents. Preferably the polymers are ones in which up to about 40%, and preferably from about 0% to about 20% by weight, of the carboxy-containing monoethylenically unsaturated monomer or monomers has been replaced by one or more non-carboxyl-containing monoethylenically unsaturated monomer or monomers containing only physiologically and ophthalmically innocuous substituents, including acrylic and methacrylic acid esters such as methyl methacrylate, ethyl acrylate, butyl acrylate, 2-ethylhexylacrylate, octyl methacrylate, 2-hydroxyethyl-methacrylate, 3-hydroxypropylacrylate, and the like, vinyl acetate, N-vinylpyrrolidone, and the like; see Mueller et al. U.S. Pat. No. 4,548,990 for a more extensive listing of such additional monoethylenically unsaturated monomers.

Particularly preferred polymers are lightly crosslinked acrylic acid polymers wherein the crosslinking monomer is 2,3-dihydroxyhexa-1,5-diene or 2,3-dimethylhexa-1,5-diene. Preferred commercially available polymers include polycarbophil (Noveon AA-1) and Carbopol®. Most preferably, a carboxy-containing polymer system known by the tradename DuraSite®, containing polycarbophil, which is a sustained release topical ophthalmic delivery system that releases the drug at a controlled rate, is used in the aqueous polymeric suspension composition of the present invention.

The crosslinked carboxy-vinyl polymers used in practicing this invention are preferably prepared by suspension or emulsion polymerizing the monomers, using conventional free radical polymerization catalysts, to a dry particle size of not more than about 50 μm in equivalent spherical diameter; e.g., to provide dry polymer particles ranging in size from about 1 to about 30 μm, and preferably from about 3 to about 20 μm, in equivalent spherical diameter. Using polymer particles that were obtained by mechanically milling larger polymer particles to this size is preferably avoided. In general, such polymers will have a molecular weight which has been variously reported as being from about 250,000 to about 4,000,000, and from 3,000,000,000 to 4,000,000,000.

In a more preferred embodiment of the invention for topical ophthalmic administration, the particles of crosslinked carboxy-vinyl polymer are monodisperse, meaning that they have a particle size distribution such that at least 80% of the particles fall within a 10 μm band of major particle size distribution. More preferably, at least 90% and most preferably at least 95%, of the particles fall within a 10 μm band of major particle size distribution. Also, a monodisperse particle size means that there is no more than 20%, preferably no more than 10%, and most preferably no more than 5% particles of a size below 1 μm. The use of a monodispersion of particles will give maximum viscosity and an increased eye residence time of the ophthalmic medicament delivery system for a given particle size. Monodisperse particles having a particle size of 30 μm and below are most preferred. Good particle packing is aided by a narrow particle size distribution.

The aqueous polymeric suspension normally contains proteasome inhibitor in an amount from about 0.05% to about 25%, preferably about 0.1% to about 20%, more preferably about 0.5% to about 15%, more preferably about 1% to about 12%, more preferably about 2% to about 10.0%, and polymeric suspending agent in an amount from about 0.05% to about 10%, preferably about 0.1% to about 5% and more preferably from about 0.2% to about 1.0% polymeric suspending agent. In the case of the above described water insoluble, water-swellable crosslinked carboxy-vinyl polymer, another preferred amount of the polymeric suspending agent is an amount from about 0.5% to about 2.0%, preferably from about 0.5% to about 1.2%, and in certain embodiments from about 0.6% to about 0.9%, based on the weight of the composition. Although referred to in the singular, it should be understood that one or 25 more species of polymeric suspending agent, such as the crosslinked carboxy-containing polymer, can be used with the total amount falling within the stated ranges. In one preferred embodiment, the composition contains about 0.6% to about 0.8% of a polycarbophil such as NOVEON AA-1.

In one embodiment, the amount of insoluble lightly crosslinked carboxy-vinyl polymer particles, the pH, and the osmotic pressure can be correlated with each other and with the degree of crosslinking to give a composition having a viscosity in the range of from about 500 to about 100,000 centipoise, and preferably from about 1,000 to about 30,000 or about 1,000 to about 10,000 centipoise, as measured at room temperature (about 25° C.) using a Brookfield Digital LVT Viscometer equipped with a number 25 spindle and a 13R small sample adapter at 12 rpm (Brookfield Engineering Laboratories Inc.; Middleboro, Mass.). Alternatively, when the viscosity is within the range of 500 to 3000 centipoise, it may be determined by a Brookfield Model DV-11+, choosing a number cp-52 spindle at 6 rpm.

When water soluble polymers are used as the suspending agent, such as hydroxypropyl methylcellulose, the viscosity will typically be about 10 to about 400 centipoise, more typically about 10 to about 200 centipoises or about 10 to about 25 centipoise.

The stabilized proteasome inhibitor formulations of the instant invention containing aqueous polymeric suspensions may be formulated so that they retain the same or substantially the same viscosity in the eye that they had prior to administration to the eye. Alternatively, in the most preferred embodiments for ocular administration, they may be formulated so that there is increased gelation upon contact with tear fluid. For instance, when a stabilized formulation containing DuraSite® or other similar polyacrylic acid-type polymer at a pH of about 5.8 to about 6.8, or more preferably about 6.0 to about 6.5, or more preferably at a pH of about 6.2 to about 6.4, or more preferably about 6.25 to about 6.35, or more preferably about 6.3 is administered to the eye, the polymer will swell upon contact with tear fluid which has a higher pH. This gelation or increase in gelation leads to entrapment of the suspended proteasome inhibitor particles, thereby extending the residence time of the composition in the eye. The proteasome inhibitor is released slowly as the suspended particles dissolve over time. All these events eventually lead to increased patient comfort and increased proteasome inhibitor contact time with the eye tissues, thereby increasing the extent of drug absorption and duration of action of the formulation in the eye. These compositions advantageously combine stability and solubility characteristics of proteasome inhibitor, which display minimal degradation and relatively high solubility in aqueous compositions at the pre-administration pH, with the advantages of the gelling composition.

The viscous gels that result from fluid eye drops typically have residence times in the eye ranging from about 2 to about 12 hours, e.g., from about 3 to about 6 hours. The agents contained in these drug delivery systems will be released from the gels at rates that depend on such factors as the drug itself and its physical form, the extent of drug loading and the pH of the system, as well as on any drug delivery adjuvants, such as ion exchange resins compatible with the ocular surface, which may also be present.

Solid Dosage Forms for Oral Use

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, preferably to delay disintegration and absorption in the gastrointestinal tract until the tablets reach the colon. The coating can be adapted to not release the proteasome inhibitor until after passage through the stomach, for example, by using an enteric coating (e.g., a pH-sensitive enteric polymer).

The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or a coating based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose. Furthermore, a time delay material such as, for example, glyceryl monostearate or glyceryl distearate, may be employed.

The solid tablet compositions may include a coating adapted to protect the composition from unwanted chemical changes (e.g., chemical degradation prior to the release of the active drug substance). The coating may be applied on the solid dosage form in a similar manner as that described in Encyclopedia of Pharmaceutical Technology.

Formulations for oral use may also be presented as hard gelatin capsules wherein the active ingredient is mixed with an inert solid diluent (e.g., potato starch, lactose, microcrystalline cellulose, calcium carbonate, calcium phosphate or kaolin). Powders and granulates may be prepared using the ingredients mentioned above under tablets and capsules in a conventional manner using, e.g., a mixer, a fluid bed apparatus or a spray drying equipment.

Controlled Release Oral Dosage Forms

Controlled release compositions for oral use may be constructed to release the active drug by controlling the dissolution and/or the diffusion of the active drug sub stance.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner. Examples include single or multiple unit tablet or capsule compositions, oil solutions, suspensions, emulsions, microcapsules, microspheres, nanoparticles, patches, and liposomes. Additional examples include the formulations listed on the following websites: http://www.advancispharm.com/, http://www.intecpharma.com/, and www.depomedinc.com/

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, camauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated metylcellulose, carnauba wax and stearyl alcohol, carbopol 934, silicone, glyceryl tristearate, methyl acrylate-methyl inethacrylate, polyvinyl chloride, polyethylene, and/or halogenated fluorocarbon.

Optional Components

In addition to the additional active agents that might be used, the compositions can also contain one or more of the following: surfactants, adjuvants including additional medicaments, buffers, antioxidants, tonicity adjusters, preservatives, thickeners or viscosity modifiers, and the like. Additives in the formulation may desirably include sodium chloride, EDTA (disodium edetate), and/or BAK (benzalkonium chloride), sorbic acid, methyl paraben, propyl paraben, and chlorhexidine. Other excipients compatible with various routes of adminsitration such as topical and parenteral administration are outlined in Remington's Pharmaceutical Sciences, Mack Publishing Co., Easton, Pa., 18.sup.th edition (1990).

### III. Optional Additional Active Agents

A further aspect of the present invention involves the above-mentioned use of additional active agents in combination with the proteasome inhibitor. A composition comprising proteasome inhibitor, an additional active agent, and an ophthalmically acceptable carrier can advantageously simplify administration and allow for treating or preventing multiple conditions or symptoms simultaneously. The “additional medicaments,” which can be present in any of the ophthalmic compositional forms described herein including fluid and solid forms, are pharmaceutically active compounds having efficacy in ocular application and which are compatible with proteasome inhibitor and with the eye.

Typically, the additional medicaments include anti-inflammatory agents including steroidal and non-steroidal anti-inflammatories, anti-allergic agents, antivirals, antifungals, and anesthetics. These other medicaments are generally present in a therapeutically effective amount. These amounts are generally within the range of from about 0.01 to 5%, more typically 0.1 to 2%, for fluid compositions and typically from 0.5 to 50% for solid dosage forms.

Anesthetics

Representative anesthetics used in ocular surgeries include tetracaine, lidocaine, marcaine, oxybuprocaine, benzocaine, butamben, dibucaine, pramoxine, proparacaine, proxymetacaine, cocaine, and Alpha-2 adrenergic receptor agonists such as Dexmedetomidine and Propofol.

Anti-Inflammatories

Steroids are one of the most commonly used medications for decreasing ocular inflammation. By inhibiting the breakdown of phospholipids into arachidonic acid, these agents act early on the inflammatory pathway. The most common side effects of this class of medications are cataract formation and glaucoma. Representative anti-inflammatory agents used for ophthalmic indications include dexamethasone, fluocinolone, loteprednol, difluprednate, fluorometholone, prednisolone, medrysone, triamcinolone, rimexolone, and pharmaceutically-acceptable salts thereof. Drugs such as loteprednol etabonate (Lotemax; Bausch+Lomb, Rochester, N.Y.) carry a lower risk of increased IOP.1 Another new agent is difluprednate (Durezol; Sirion Therapeutics, Tampa, Fla.), which possesses even greater potency than the other available corticosteroids.

Although nonsteroidal anti-inflammatory drugs have been used to treat inflammatory conditions, physicians should exercise caution when prescribing this class of medications. In patients with severe inflammation combined with dry eye disease, treatment with non-steroidal anti-inflammatory drugs has caused corneal melting (Isawi and Dhaliwal, “Corneal melting and perforation in Stevens Johnson syndrome following topical bromfenac use,” J Cataract Refract Surg. 2007; 33(9):1644-1646). In contrast, cyclosporine 0.05% (Restasis; Allergan, Inc., Irvine, Calif.) has been shown to effectively control many causes of ocular surface inflammation, and this ophthalmic emulsion has an excellent safety profile. Accordingly, combinations of a proteasome inhibitor and cyclosporine, particularly in the form of ocular formulations such as eye drops, are also within the scope of the invention. Representative non-steroidal anti-inflammatory agents used in ophthalmic indications include Acular, Acular LS, Acuvail, Bromday, bromfenac, diclofenac, flurbiprofen, Ilevro, ketorolac, nepafenac, Nevanac, Ocufen, Prolensa, and Voltaren.

Combination Therapy

Because ocular disorders are frequently associated with inflammation, it can be advantageous to co-administer the proteasome inhibitor with one or more anti-inflammatory agents. One such combination includes both proteasome inhibitor and dexamethasone, which can be administered in the form of a suspension, or in the form of eye drops, for topical application. Another representative corticosteroid is loteprednol etabonate.

The combination therapy can be extremely useful in connection with steroid-responsive inflammatory ocular conditions for which a corticosteroid is indicated and where bacterial infection or a risk of bacterial ocular infection exists.

Ocular steroids are indicated in inflammatory conditions of the palpebral and bulbar conjunctiva, cornea, and anterior segment of the globe, where the inherent risk of steroid use in certain infective conjunctivitis is accepted to obtain a diminution in edema and inflammation. They are also indicated in chronic anterior uveitis and corneal injury from chemical, radiation or thermal burns, or penetration of foreign bodies.

The use of a combination drug product that includes a proteasome inhibitor and an anti-inflammatory agent is indicated where the risk of inflammation is high. Steroids are one of the most commonly used medications for decreasing ocular inflammation. By inhibiting the breakdown of phospholipids into arachidonic acid, these agents act early on the inflammatory pathway. The most common side effects of this class of medications are cataract formation and glaucoma. Drugs such as loteprednol etabonate (Lotemax; Bausch+Lomb, Rochester, N.Y.) carry a lower risk of increased TOP. Another new agent is difluprednate (Durezol; Sirion Therapeutics, Tampa, Fla.), which possesses even greater potency than the other available corticosteroids.

Although nonsteroidal anti-inflammatory drugs have been used to treat inflammatory conditions, physicians should exercise caution when prescribing this class of medications. In patients with severe inflammation combined with dry eye disease, treatment with non-steroidal anti-inflammatory drugs has caused corneal melting (Isawi and Dhaliwal, “Corneal melting and perforation in Stevens Johnson syndrome following topical bromfenac use,” J Cataract Refract Surg. 2007; 33(9):1644-1646). In contrast, cyclosporine 0.05% (Restasis; Allergan, Inc., Irvine, Calif.) has been shown to effectively control many causes of ocular surface inflammation, and this ophthalmic emulsion has an excellent safety profile. Accordingly, combinations of a proteasome inhibitor and cyclosporine, particularly in the form of ocular formulations such as eye drops, are also within the scope of the invention.

If additional therapy is required, autologous serum tears can be very effective. Because they contain several important components of natural tears such as epidermal growth factor, fibronectin, and vitamin A, autologous serum tears increase the health of the ocular surface (Kojima, et al., Autologous serum eye drops for the treatment of dry eye diseases, Cornea, 27(suppl 1):S25-30 (2008)).

Another alternative is to use agents such as tacrolimus, sirolimus, and the like, for example, in the form of a dermatologic ointment (Protopic; Astellas Pharma US, Inc., Deerfield, Ill.) (Wyrsch et al., “Safety of treatment with tacrolimus ointment for anterior segment inflammatory diseases,” Klin Monatsbl Augenheilkd, 226(4):234-236 (2009)). Thus, combinations of these agents and a proteasome inhibitor as described herein are also within the scope of the invention.

Combinations of Proteasome Inhibitors and Antimicrobial Agent

The proteasome inhibitors described herein can be administered before, during, or after administration of an antimicrobial agent, and an antimicrobial compound can be included in the proteasome inhibitor-containing compositions. The antimicrobials include antibiotics, antivirals, and antifungals.

Exemplary antibiotics include beta-lactams such as penicillins (e.g., penicillin G, penicillin V, methicillin, oxacillin, cloxacillin, dicloxacillin, nafcillin, ampicillin, amoxicillin, carbenicillin, ticarcillin, mezlocillin, piperacillin, azlocillin, and temocillin), cephalosporins (e.g., cepalothin, cephapirin, cephradine, cephaloridine, cefazolin, cefamandole, cefuroxime, cephalexin, cefprozil, cefaclor, loracarbef, cefoxitin, cefmatozole, cefotaxime, ceftizoxime, ceftriaxone, cefoperazone, ceftazidime, cefixime, cefpodoxime, ceftibuten, cefdinir, cefpirome, cefepime, BAL5788, and BAL9141), carbapenams (e.g., imipenem, ertapenem, and meropenem), and monobactams (e.g., astreonam); beta-lactamase inhibitors (e.g., clavulanate, sulbactam, and tazobactam); aminoglycosides (e.g., streptomycin, neomycin, kanamycin, paromycin, gentamicin, tobramycin, amikacin, netilmicin, spectinomycin, sisomicin, dibekalin, and isepamicin); tetracyclines (e.g., tetracycline, chlortetracycline, demeclocycline, minocycline, oxytetracycline, methacycline, and doxycycline); macrolides (e.g., erythromycin, azithromycin, and clarithromycin); ketolides (e.g., telithromycin, ABT-773); lincosamides (e.g., lincomycin and clindamycin); glycopeptides (e.g., vancomycin, oritavancin, dalbavancin, and teicoplanin); streptogramins (e.g., quinupristin and dalfopristin); sulphonamides (e.g., sulphanilamide, para-aminobenzoic acid, sulfadiazine, sulfisoxazole, sulfamethoxazole, and sulfathalidine); oxazolidinones (e.g., linezolid); quinolones (e.g., nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, gatifloxacin, moxifloxacin, gemifloxacin, and sitafloxacin); metronidazole; daptomycin; garenoxacin; ramoplanin; faropenem; polymyxin; tigecycline, AZD2563; and trimethoprim.

These antibiotics can be used in the dose ranges currently known and used for these agents, particularly when such are prescribed for treating ocular disorders. Different concentrations may be employed depending on the clinical condition of the patient, the goal of therapy (treatment or prophylaxis), the anticipated duration, and the severity of the infection for which the drug is being administered. Additional considerations in dose selection include the type of infection, age of the patient (e.g., pediatric, adult, or geriatric), general health, and co-morbidity. Determining what concentrations to employ are within the skills of the pharmacist, medicinal chemist, or medical practitioner. Typical dosages and frequencies are provided, e.g., in the Merck Manual of Diagnosis & Therapy (17th Ed. M H Beers et al., Merck & Co.).

### IV. Treatment of Ocular Disorders

The proteasome inhibitors described herein are suitable for use in treating ocular disorders mediated by proteasomes, and ocular disorders associated with inflammation, including those resulting from a bacterial, viral or fungal infection.

Ocular Disorders with an Inflammatory Component

Several ocular disorders have an inflammatory component, and thus can be treated or prevented using the proteasome inhibitors described herein. Representative types of inflammatory ocular disorders that can be treated using the proteasome inhibitors described herein, for example, by topical application of compositions including one or more proteasome inhibitor, and also optionally including an anti-inflammatory agent, include ocular rosacea, wet and dry age-related macular degeneration (AMD), diabetic retinopathy (DR), glaucoma, neovascular glaucoma, retinal vasculitis, uveitis, such as posterior uveitis, keratoconjunctivitis sicca, conjunctivitis, retinitis secondary to glaucoma, neovascular glaucoma, episcleritis, scleritis, optic neuritis, retrobulbar neuritis, ocular inflammation following ocular surgery, ocular inflammation resulting from physical eye trauma, Pterygium (Surfer's Eye), cataract, ocular allergy, dry eye, blepharitis, meibomian gland dysfunction, neurodegenerative disorders affecting the retina, including Alzheimer's, Parkinson's, and Huntington's diseases, and other retina-specific illnesses with UPS involvement, such as retinitis pigmentosa and age-related impairments.

Specific ocular disorders are described in more detail below.

Ocular Rosacea

In one embodiment, the ocular disorder to be treated or prevented is ocular rosacea. Ocular rosacea is a manifestation of rosacea that affects the eyes and eyelids. Signs and symptoms generally consist of redness, irritation or burning of the eyes. Affected individuals may also feel that there is something, such as an eyelash, in the eye and frequently have redness of the nose and cheeks as well.

Tear film disturbances are responsible for the vast majority of subjective complaints and objective findings in ocular rosacea. The reduced amount and altered character of meibomian gland secretions result in destabilization of the lipid portion of the tear film and increased tear evaporation rates. More than one-third of patients with rosacea also have impaired aqueous tear secretion, further contributing to ocular surface desiccation.

It is believed that some complications of ocular rosacea may result from reactions of the sclera, limbus and cornea to staphylococcal endotoxins or cell-mediated hypersensitivity responses to staphylococcal antigens. The variability in response of patients with ocular rosacea to these immune reactions may account for the extreme variability in clinical signs and symptoms associated with this disorder.

Conventional therapy for ocular rosacea includes normalizing tear film disturbances, typically with warm compresses, punctal occlusion (temporary or permanent) if aqueous tear production is deficient, using artificial tears to help provide ocular surface wetting, controlling bacterial overgrowth, including keeping the eyelids clean, using topical antibiotics to reduce bacterial flora, particularly when acute mucopurulent blepharoconjunctivitis, marginal corneal infiltrates or peripheral ulcerative keratitis are present, and controlling inflammatory and hypersensitivity reactions, for example, using topical corticosteroids, including topical progestational steroids such as compounded medroxyprogesterone (typically at around a 1% concentration by weight).

Any of these conventional approaches can be combined with treatment using proteasome inhibitors. For example, a proteasome inhibitor can be added to an artificial tears formulation, and a steroid and/or an antibiotic can also be added.

Age-Related Macular Degeneration

Macular degeneration is the leading cause of severe vision loss in people over age 60. It occurs when the small central portion of the retina, known as the macula, deteriorates. The retina is the light-sensing nerve tissue at the back of the eye. Because the disease develops as a person ages, it is often referred to as age-related macular degeneration (AMD). Although macular degeneration is almost never a totally blinding condition, it can be a source of significant visual disability.

There are two main types of age-related macular degeneration:

Dry form. The “dry” form of macular degeneration is characterized by the presence of yellow deposits, called drusen, in the macula. A few small drusen may not cause changes in vision; however, as they grow in size and increase in number, they may lead to a dimming or distortion of vision that people find most noticeable when they read. In more advanced stages of dry macular degeneration, there is also a thinning of the light-sensitive layer of cells in the macula leading to atrophy, or tissue death. In the atrophic form of dry macular degeneration, patients may have blind spots in the center of their vision. In the advanced stages, patients lose central vision.

Wet form. The “wet” form of macular degeneration is characterized by the growth of abnormal blood vessels from the choroid underneath the macula. This is called choroidal neovascularization. These blood vessels leak blood and fluid into the retina, causing distortion of vision that makes straight lines look wavy, as well as blind spots and loss of central vision. These abnormal blood vessels eventually scar, leading to permanent loss of central vision.

Non-Infectious Anterior Uveitis

One example of an ocular disorder associated with inflammation is noninfectious anterior uveitis. This disorder is typically treated using corticosteroids such as prednisolone acetate (0.125% and 1% by weight), Betamethasone (1% by weight), Dexamethasone sodium phosphate (0.1% by weight in eye drops, 0.05% by weight in ointment form), Fluorometholone (0.1% and 0.25% by weight, or 0.1% in ointment form), Loteprednol, and Rimexolone (1% by weight).

The choice of topical steroid is typically made by the treating physician with respect to the severity of uveitis. Topical non-steroidal anti-inflammatory drugs (NSAIDs) like flubriprofen can also be used.

Ocular Disorders Caused by or Associated with Microbial Infection

Certain ocular disorders have a microbial component, including viruses, bacteria, fungi, and parasites.

The proteasome inhibitor formulations of this invention can be used, along with an appropriate antimicrobial agent, to treat or prevent a variety of conditions associated with ocular infection. The role of the proteasome inhibitor in this setting is to minimize damage associated with inflammation, while the antimicrobial agent is administered to address the underlying cause of the inflammation (i.e., the microbial infection).

For example, conditions of the eyelids, including blepharitis, blepharconjunctivies, meibomianitis, acute or chronic hordeolum, chalazion, dacryocystitis, dacryoadenities, and acne rosacea; conditions of the conjunctiva, including conjunctivitis, ophthalmia neonatorum, and trachoma; conditions of the cornea, including corneal ulcers, superficial and interstitial keratitis, keratoconjunctivitis, foreign bodies, and post operative infections; and conditions of the anterior chamber and uvea, including endophthalmitis, infectious uveitis, and post operative infections, are a few of the tissues and conditions that can be treated by topical application of the proteasome inhibitor and the antimicrobial agent.

The prevention of infection includes pre-operative treatment prior to surgery as well as other suspected infectious conditions or contact. Examples of prophylaxis situations include treatment prior to surgical procedures such as blepharoplasty, removal of chalazia, tarsorrhapy, procedures for the canualiculi and lacrimal drainage system and other operative procedures involving the lids and lacrimal apparatus; conjunctival surgery including removal of ptyregia, pingueculae and tumors, conjunctival transplantation, traumatic lesions such as cuts, burns and abrasions, and conjunctival flaps; corneal surgery including removal of foreign bodies, keratotomy, and corneal transplants; refractive surgery including photorefractive procedures; glaucoma surgery including filtering blebs; paracentesis of the anterior chamber; iridectomy; cataract surgery; retinal surgery; and procedures involving the extraocular muscles. The prevention of ophthalmia neonatorum is also included.

The compositions described herein, including a proteasome inhibitor and an appropriate antimicrobial agent specific for the type of microbial infection, can be used to treat or prevent an ocular infection, and to prevent, minimize, or treat inflammation resulting from an ocular infection.

Specific indications that can be treated or prevented include conditions of the eyelids, including blepharitis, blepharconjunctivies, meibomianitis, acute or chronic hordeolum, chalazion, dacryocystitis, dacryoadenities, and acne rosacea; conditions of the conjunctiva, including conjunctivitis, ophthalmia neonatorum, and trachoma; conditions of the cornea, including corneal ulcers, superficial and interstitial keratitis, keratoconjunctivitis, foreign bodies, and post operative infections; and conditions of the anterior chamber and uvea, including endophthalmitis, infectious uveitis, and post operative infections.

The prevention of infection includes pre-operative treatment prior to surgery as well as other suspected infectious conditions or contact. Examples of prophylaxis situations include treatment prior to surgical procedures such as blepharoplasty, removal of chalazia, tarsorrhapy, procedures for the canualiculi and lacrimal drainage system and other operative procedures involving the lids and lacrimal apparatus; conjunctival surgery including removal of ptyregia, pingueculae and tumors, conjunctival transplantation, traumatic lesions such as cuts, burns and abrasions, and conjunctival flaps; corneal surgery including removal of foreign bodies, keratotomy, and corneal transplants; refractive surgery including photorefractive procedures; glaucoma surgery including filtering blebs; paracentesis of the anterior chamber; iridectomy; cataract surgery; retinal surgery; and procedures involving the extraocular muscles. The prevention of ophthalmia neonatorum is also included.

Representative microbial species include one or more of the following organisms: Staphylococcus including Staphylococcus aureus and Staphylococcus epidermidis; Streptococcus including Streptococcus pneumoniae and Streptococcus pyogenes as well as Streptococci of Groups C, F, and G and Viridans group of Streptococci; Haemophilus influenza including biotype III (H. Aegyptius); Haemophilus ducreyi; Moraxella catarrhalis; Neisseria including Neisseria gonorrhoeae and Neisseria meningitidis; Chlamydia including Chlamydia trachomatis, Chlamydia psittaci, and Chlamydia pneumoniae; Mycobacterium including Mycobacterium tuberculosis and Mycobacterium avium-intracellular complex as well as a typical mycobacterium including M. marinum, M. fortuitm, and M. chelonae; Bordetella pertussis; Campylobacter jejuni; Legionella pneumophila; Bacteroides bivius; Clostridium perfringens; Peptostreptococcus species; Borrelia burgdorferi; Mycoplasma pneumoniae; Treponema pallidum; Ureaplasma urealyticum; toxoplasma; malaria; and nosema.

Some of the more common genera found are Haemophilus, Neisseria, Staphylococcus, Streptococcus, and Chlamydia. Specific types of ocular disorders that can be treated or prevented by the active agents-containing compositions include, but are not limited to, the following:

Trachoma

Trachomatis is an infectious eye disease, and the leading cause of the world's infectious blindness. Globally, 84 million people suffer from active infection and nearly 8 million people are visually impaired as a result of this disease.

Trachoma is caused by Chlamydia trachomatis and it is spread by direct contact with eye, nose, and throat secretions from affected individuals, or contact with fomites (inanimate objects), such as towels and/or washcloths, that have had similar contact with these secretions. Flies can also be a route of mechanical transmission. Untreated, repeated trachoma infections result in entropion—a painful form of permanent blindness when the eyelids turn inward, causing the eyelashes to scratch the cornea.

The bacterium has an incubation period of 5 to 12 days, after which the affected individual experiences symptoms of conjunctivitis, or irritation similar to “pink eye.” Blinding endemic trachoma results from multiple episodes of re-infection that maintains the intense inflammation in the conjunctiva. Without re-infection, the inflammation will gradually subside.

The conjunctival inflammation is called “active trachoma” and usually is seen in children, especially pre-school children. It is characterized by white lumps in the undersurface of the upper eye lid (conjunctival follicles or lymphoid germinal centers) and by non-specific inflammation and thickening often associated with papillae. Follicles may also appear at the junction of the cornea and the sclera (limbal follicles). Active trachoma will often be irritating and have a watery discharge. Bacterial secondary infection may occur and cause a purulent discharge.

The later structural changes of trachoma are referred to as “cicatricial trachoma”. These include scarring in the eye lid (tarsal conjunctiva) that leads to distortion of the eye lid with buckling of the lid (tarsus) so the lashes rub on the eye (trichiasis). These lashes will lead to corneal opacities and scarring, and then to blindness.

The compositions described herein can be used prophylactically to prevent the spread of infection, and/or to prevent the onset of symptoms associated with inflammation. Prophylactic administration can be used, for example, in poor communities where infection has already occurred, and is likely to spread.

In one embodiment, one can administer drops of the stabilized solutions described herein to the eyes of individuals suffering from, or at risk from suffering from, a C. trachomatis infection in their eyes.

Bacterial Conjunctivitis

Bacterial conjunctivitis is a purulent infection of the conjunctiva by any of several species of gram-negative, gram-positive, or acid-fast organisms. Some of the more commonly found genera causing conjunctival infections are Haemophilus, Streptococcus, Neisseria, and Chlamydia.

Hordeolum

Hordeolum is a purulent infection of one of the sebaceous glands of Zeis along the eyelid margin (external) or of the meibomian gland on the conjunctival side of the eyelid (internal).

Infectious Keratoconjunctivitis

Infectious keratoconjunctivitis is an infectious disease of cattle, sheep, and goats, characterized by blepharospasm, lacrimation, conjunctivitis, and varying degrees of corneal opacity and ulceration. In cattle the causative agent is Moraxella bovis; in sheep, mycoplasma, rickettsia, Chlamydia, or acholeplasma, and in goats, rickettsia.

Ocular Tuberculosis

Ocular tuberculosis is an infection of the eye, primarily the iris, ciliary body, and choroid.

Uveitis

Uveitis is the inflammatory process that involves the uvea or middle layers of the eye. The uvea includes the iris (the colored part of the eye), the choroid (the middle blood vessel layer) and the ciliary body—the part of the eye that joins both parts. Uveitis is the eye's version of arthritis. The most common symptoms and signs are redness in the white part of the eye, sensitivity to light, blurry vision, floaters, and irregular pupil. Uveitis can present at any age, including during childhood.

Uveitis is easily confused with many eye inflammations, such as conjunctivitis (conjunctival inflammation) or pink eye; keratitis (corneal inflammation); episleritis or scleritis (blood vessel inflammation in the episclera or sclera respectively); or acute closed angle glaucoma.

Suppurative Uveitis

Suppurative uveitis is an intraocular infection caused mainly by pus-producing bacteria, and rarely by fungi. The infection may be caused by an injury or surgical wound (exogenous) or by endogenous septic emboli in such diseases as bacterial endocarditis or meningococcemia.

Blepharatis

Nonspecific conjunctivitis (NSC) has many potential causes, including fatigue and strain, environmental dryness and pollutants, wind and dust, temperature and radiation, poor vision correction, contact lens use, computer use and dry eye syndrome. Another cause relates to the body's innate reaction to dead cells, which can cause nonspecific conjunctivitis.

This type of infection can occur when a patient's lid disease causes mild conjunctivitis, and dead Staphylococcal bacteria from the lids fall onto the ocular surface. The cells trigger an inflammatory hypersensitivity reaction on the already irritated eyes. This inflammatory reaction against the dead cells can be treated using the proteasome inhibitors described herein, optionally in combination with another anti-inflammatory agent, to combat inflammation, and an antibacterial compound to address the underlying cause of the inflammation, namely, infection by living Staph bacteria.

Aside from allergy, the combined causes of inflammation and infection are probably the most common origins of conjunctivitis. In fact, this combination is more common than all types of infection combined. The concentration of mast cells in the conjunctiva and the eyelids makes them prime targets for hypersensitivity reactions and inflammatory disease. A compromised ocular surface cannot protect itself from bacteria with full efficacy. Although NSC patients may not have full-blown bacterial infections, their eyes are susceptible to some bacterial disease components.

Unlike patients with allergic conjunctivitis, who are typically treated using steroids alone, or patients who need a strong antibiotic for bacterial disease, NSC patients can benefit from a combination treatment (active agents and an anti-inflammatory agent) to battle inflammatory NSC.

Corneal Inflammation

Corneal inflammation is one of the most common ocular diseases in both humans and animals and can lead to blindness or even cause lost of the eye itself. In humans, keratitis is classified into infectious and non-infectious, while in veterinary medicine the tradition is to classify keratitis rather into ulcerative and non-ulcerative (Whitley and Gilger 1999). Non-ulcerative keratitis in dogs is usually caused by mechanical irritation (pigmentary keratitis) or by immune-mediated process (pannus). However, non-ulcerative infectious keratitis also exists (corneal abscess, mycotic, viral keratitis). Ulcerative keratitis can be of non-infectious (recurrent erosions, traumatically induced superficial ulceration) or infectious (bacterial, viral, mycotic) origin. Even in the cases of originally non-infectious ulceration, after disruption in the epithelium secondary infection often occurs.

Parasitic Eye Infections

There are also a variety of ocular infestations caused by parasites like brucellosis. For example, toxocara infections can cause ocular larva migrans (OLM), an eye disease that can cause blindness. OLM occurs when a microscopic worm enters the eye; it may cause inflammation and formation of a scar on the retina. Cysticercosis is a parasitic infestation of different body organs by Cysticercosis cellulosae. Ocular manifestations of malaria and leishmaniasis are well documented and sight threatening conditions.

These and other ocular parasitic infections can be treated by using the compounds described herein to treat the inflammation, and treating the underlying disorder with an appropriate anti-parasitic agent.

### IV. Methods of Treating or Preventing Inflammation Following Ocular Surgery

Following eye surgery, a patient may suffer from ocular inflammation. Administration of a proteasome inhibitor as described herein, before, during, and/or after eye surgery, can minimize, prevent, or treat the inflammation. Representative eye surgeries for which administration of proteasome inhibitors can be used include, but are not limited to, the following.

Laser Eye Surgery

Laser eye surgery can be used to treat non-refractive conditions (for example, to seal a retinal tear), while radial keratotomy is an example of refractive surgery that can be performed without using a laser.

Laser eye surgery, or laser corneal sculpting, is a medical procedure that uses a laser to reshape the surface of the eye to improve or correct myopia (short-sightedness), hypermetropia (long sightedness) and astigmatism (uneven curvature of the eye's surface).

Refractive Surgery

Refractive surgery aims to correct errors of refraction in the eye, reducing or eliminating the need for corrective lenses. Also, limbal relaxing incisions (LRI) can be used to correct minor astigmatism.

Keratoplasy and Keratotomy

Keratoplasty is defined as surgery performed upon the cornea, such as a corneal transplantation/grafting.

Keratotomy is a type of refractive surgical procedure, and can refer to radial keratotomy or photorefractive keratotomy.

Examples include astigmatic keratotomy (AK), also known as arcuate keratotomy or transverse keratotomy, radial keratotomy (RK), Mini Asymmetric Radial Keratotomy (M.A.R.K.), which involves preparing a series of microincisions to cause a controlled cicatrisation of the cornea, which changes its thickness and shape and can correct astigmatism and cure the first and second stage of keratoconus, and hexagonal keratotomy (HK).

Keratomilleusis

Keratomilleusis is a method of reshaping the cornea surface to change its optical power. A disc of cornea is shaved off, quickly frozen, lathe-ground, then returned to its original power. A variation of this type of operation is laser-assisted in-situ keratomileusis (LASIK), including laser-assisted sub-epithelial keratomileusis (LASEK), also known as Epi-LASIK. Similar procedures include IntraLASIK, automated lamellar keratoplasty (ALK), photorefractive keratectomy (PRK), laser thermal keratoplasty (LTK), and conductive keratoplasty (CK), which uses radio frequency waves to shrink corneal collagen and is used to treat mild to moderate hyperopia.

Cataract Surgery

A cataract is an opacification or cloudiness of the eye's crystalline lens that prevents light from forming a clear image on the retina. If visual loss is significant, surgical removal of the lens may be warranted, with lost optical power usually replaced with a plastic intraocular lens (IOL).

Glaucoma Surgery

Glaucoma is a group of diseases affecting the optic nerve that results in vision loss and is frequently characterized by raised intraocular pressure (TOP). There are many types of glaucoma surgery, and variations or combinations of those types, that facilitate the escape of excess aqueous humor from the eye to lower intraocular pressure, and a few that lower TOP by decreasing the production of aqueous humor.

Canaloplasty

Canaloplasty enhances drainage through the eye's natural drainage system to provide sustained reduction of intra-ocular pressure (TOP). Canaloplasty uses microcatheter technology to create a tiny incision to gain access to a canal in the eye. The microcatheter circumnavigates the canal around the iris, enlarging the main drainage channel and its smaller collector channels through the injection of a sterile, gel-like material called viscoelastic. The catheter is then removed and a suture is placed within the canal and tightened. By opening up the canal, the pressure inside the eye can be reduced.

Karmra Inlays

A Karmra inlay is placed inside the cornea, and has a small aperture that gives clearer vision at intermediate and near distances.

Scleral Reinforcement Surgery

Scleral reinforcement surgery is used to mitigate degenerative myopia.

Corneal Surgery

Corneal surgery includes most refractive surgery, as well as corneal transplant surgery, penetrating keratoplasty (PK), keratoprosthesis (KPro), phototherapeutic keratectomy (PTK), pterygium excision, corneal tattooing, and osteo-odonto-keratoprosthesis (OOKP), in which support for an artificial cornea is created from a tooth and its surrounding jawbone.

Vitreo-Retinal Surgery

Vitreo-retinal surgery includes vitrectomies, including anterior vitrectomy, which removes the front portion of vitreous tissue to prevent or treat vitreous loss during cataract or corneal surgery, or to remove misplaced vitreous in conditions such as aphakia pupillary block glaucoma.

Pars plana vitrectomy (PPV), or trans pars plana vitrectomy (TPPV), removes vitreous opacities and membranes through a pars plana incision, and is frequently combined with other intraocular procedures for the treatment of giant retinal tears, tractional retinal detachments, and posterior vitreous detachments.

Pan retinal photocoagulation (PRP) is a type of photocoagulation therapy used in the treatment of diabetic retinopathy.

Retinal Detachment Repair

A scleral buckle is often used to repair a retinal detachment to indent or “buckle” the sclera inward, usually by sewing a piece of preserved sclera or silicone rubber to its surface. Laser photocoagulation, or photocoagulation therapy, involves using a laser to seal a retinal tear.

Pneumatic Retinopexy

Retinal cryopexy, or retinal cryotherapy, is a procedure that uses intense cold to induce a chorioretinal scar and to destroy retinal or choroidal tissue.

Eye Muscle Surgery

Eye muscle surgery typically corrects strabismus and includes transposition/repositioning procedures, tightening/strengthening procedures, loosening/weakening procedures, advancement (moving an eye muscle from its original place of attachment on the eyeball to a more forward position), recession (moving the insertion of a muscle posteriorly towards its origin), myectomy, myotomy, tenectomy, tenotomy, resection, tucking, isolating the inferior rectus muscle, and disinserting the medial rectus muscle.

Adjustable suture surgery involves reattaching an extraocular muscle using a stitch that can be shortened or lengthened within the first post-operative day, to obtain better ocular alignment.

Surgery Involving the Lacrimal Apparatus

A dacryocystorhinostomy (DCR) or dacryocystorhinotomy restores the flow of tears into the nose from the lacrimal sac when the nasolacrimal duct does not function.

Canaliculodacryocystostomy is a surgical correction for a congenitally blocked tear duct in which the closed segment is excised and the open end is joined to the lacrimal sac.

Canaliculotomy involves slitting of the lacrimal punctum and canaliculus for the relief of epiphora.

A dacryoadenectomy is the surgical removal of a lacrimal gland.

A dacryocystectomy is the surgical removal of a part of the lacrimal sac.

A dacryocystostomy is an incision into the lacrimal sac, usually to promote drainage.

A dacryocystotomy is an incision into the lacrimal sac.

Eye removal includes enucleation, which involves removing the eye, leaving the eye muscles and remaining orbital contents intact, evisceration, which involves removing the eye's contents, leaving the scleral shell intact (usually performed to reduce pain in a blind eye), and exenteration, which involves removing the entire orbital contents, including the eye, extraocular muscles, fat, and connective tissues (usually performed to remove malignant orbital tumors).

Other Ocular Surgical Techniques

Additional surgeries include posterior sclerotomy, in which an opening is made into the vitreous through the sclera, as for detached retina or the removal of a foreign body, macular hole repair, partial lamellar sclerouvectomy, partial lamellar sclerocyclochoroidectomy, partial lamellar sclerochoroidectomy, radial optic neurotomy, macular translocation surgery, through 360 degree retinotomy, and through scleral imbrication technique.

Epikeratophakia is the removal of the corneal epithelium and replacement with a lathe cut corneal button.

Implants can be inserted, including intracorneal rings (ICRs), corneal ring segments (Intacs), implantable contact lenses, and scleral expansion bands (SEB).

Presbyopia is a condition where, with age, the eye exhibits a progressively diminished ability to focus on near objects. Presbyopia can be reversed surgically, including through anterior ciliary sclerotomy (ACS), and laser reversal of presbyopia (LRP).

A ciliarotomy is a surgical division of the ciliary zone in the treatment of glaucoma.

A ciliectomy is 1) the surgical removal of part of the ciliary body, or 2) the surgical removal of part of a margin of an eyelid containing the roots of the eyelashes.

A ciliotomy is a surgical section of the ciliary nerves.

A conjunctivoanstrostomy is an opening made from the inferior conjuctival cul-de-sac into the maxillary sinus for the treatment of epiphora.

Conjuctivoplasty is plastic surgery of the conjunctiva.

A conjunctivorhinostomy is a surgical correction of the total obstruction of a lacrimal canaliculus by which the conjuctiva is anastomosed with the nasal cavity to improve tear flow.

A corectomedialysis, or coretomedialysis, is an excision of a small portion of the iris at its junction with the ciliary body to form an artificial pupil.

A corectomy, or coretomy, is any surgical cutting operation on the iris at the pupil.

A corelysis is a surgical detachment of adhesions of the iris to the capsule of the crystalline lens or cornea.

A coremorphosis is the surgical formation of an artificial pupil.

A coreplasty, or coreoplasty, is plastic surgery of the iris, usually for the formation of an artificial pupil.

A coreoplasy, or laser pupillomydriasis, is any procedure that changes the size or shape of the pupil.

A cyclectomy is an excision of portion of the ciliary body.

A cyclotomy, or cyclicotomy, is a surgical incision of the ciliary body, usually for the relief of glaucoma.

A cycloanemization is a surgical obliteration of the long ciliary arteries in the treatment of glaucoma.

An iridectomesodialsys is the formation of an artificial pupil by detaching and excising a portion of the iris at its periphery.

An iridodialysis, sometimes known as a coredialysis, is a localized separation or tearing away of the iris from its attachment to the ciliary body.

An iridencleisis, or corenclisis, is a surgical procedure for glaucoma in which a portion of the iris is incised and incarcerated in a limbal incision.

An iridesis is a surgical procedure in which a portion of the iris is brought through and incarcerated in a corneal incision in order to reposition the pupil.

An iridocorneosclerectomy is the surgical removal of a portion of the iris, the cornea, and the sclera.

An iridocyclectomy is the surgical removal of the iris and the ciliary body.

An iridocystectomy is the surgical removal of a portion of the iris to form an artificial pupil.

An iridosclerectomy is the surgical removal of a portion of the sclera and a portion of the iris in the region of the limbus for the treatment of glaucoma.

An iridosclerotomy is the surgical puncture of the sclera and the margin of the iris for the treatment of glaucoma.

A rhinommectomy is the surgical removal of a portion of the internal canthus.

A trepanotrabeculectomy is used to treat chronic open and chronic closed angle glaucoma.

Any and all of the disorders discussed above can be treated using the proteasome inhibitors described herein, alone or in combination with other active agents, such as anti-inflammatory agents, antimicrobials, and anesthetics, using appropriate compositions as described herein.

The present invention will be better understood with reference to the following non-limiting examples. In these examples, all of the percentages recited herein refer to weight percent, unless otherwise indicated.

### Example 1

Hydroxypropylmethyl cellulose, sodium chloride, edetate sodium (EDTA), BAK and surfactant are dissolved in a beaker containing approximately ⅓ of the final weight of water and stirred for 10 minutes with an overhead stirring. Proteasome inhibitor is added and stirred to disperse for 30 minutes. The solution is sterilized by autoclaving at 121° C. for 20 minutes. Alternately, the proteasome inhibitor may be dry heat sterilized and added by aseptic powder addition after sterilization. Mannitol, Poloxamer 407, and boric acid are dissolved separately in approximately ½ of the final weight of water and added by sterile filtration (0.22 μm filter) and stirred for 10 minutes to form a mixture. The mixture is adjusted to the desired pH in the range of 5.8 to 7.0 with sterile sodium hydroxide (1N to 10N) while stirring, brought to a final weight with sterile water, and aseptically transferred to multi-dose containers.

### Example 2

Noveon AA-1, an acrylic acid polymer available from B. F. Goodrich, is slowly dispensed into a beaker containing approximately ⅓ of the final weight of water and stirred for 1.5 hrs. with an overhead stirrer. Ethylene diamine tetra acetic acid (EDTA), benzalkonium chloride (BAK), sodium chloride, and surfactant are then added to the polymer solution and stirred for 10 minutes after each addition. The polymer suspension is at a pH of about 3.0-3.5. The proteasome inhibitor is added and stirred to disperse for 30 minutes. The pH of the mixture is titrated to the desired pH in the range of 5.8 to 6.8, and brought to final weight/volume with water. The mixture is aliquoted into single or multiple dose containers, which are sterilized by autoclaving at 121° C., for 20 minutes. Alternately, the proteasome inhibitor may be dry heat sterilized and added by aseptic powder addition after sterilization. In the alternative embodiment Noveon AA-1 is slowly dispensed into a beaker containing approximately ⅓ of the final weight of water and stirred for 1.5 hrs., with overhead stirring, to form a Noveon suspension. The Noveon suspension is sterilized by autoclaving at 121° C., for 20 minutes. Solutions containing mannitol and boric acid, or solutions containing Dequest® (a brand of bisphosphonate), mannitol, and boric acid are dissolved separately in approximately ½ of the final weight of water, added to the sterilized Noveon polymer suspension by sterile filtration (0.22 μm filter), and stirred for 10 minutes. The dry heat sterilized proteasome inhibitor is then added by aseptic powder addition. The mixture is adjusted to the desired pH with sterile sodium hydroxide (1N to 10N) while stirring, brought to final weight with sterile water, and aseptically filled into multi-dose containers.

### Example 3

Noveon AA-1 is slowly dispensed into a beaker containing approximately ½ of the final weight of water and stirred for 1.5 hrs., with overhead stirring. Edetate sodium (EDTA), Poloxamer 407 (a hydrophilic non-ionic surfactant of the more general class of copolymers known as poloxamers, specifically, a triblock copolymer consisting of a central hydrophobic block of polypropylene glycol flanked by two hydrophilic blocks of polyethylene glycol, with approximate lengths of the two PEG blocks of 101 repeat units, and an approximate length of the propylene glycol block of 56 repeat units), and sodium chloride are then added to the polymer suspension and stirred for 10 minutes. The polymer suspension is at a pH of about 3.0-3.5. The proteasome inhibitor is added and stirred to disperse for 30 minutes. The mixture is adjusted to desired pH with sodium hydroxide (1N to 10N) while stirring, and is sterilized by autoclaving at 121° C. for 20 minutes. Alternately, the proteasome inhibitor may be dry heat sterilized and added by aseptic powder addition after sterilization. Mannitol is dissolved in 1/10 of the final weight of water and sterile filtered (0.22 μm filter) in to the polymer suspension and stirred for 10 minutes. The mixture is adjusted to desired pH with sterile sodium hydroxide (1N to 10N) while stirring, brought to final weight with sterile water, and aseptically filled into unit-dose containers.

### Example 4

A proteasome inhibitor ointment is prepared by dissolving 0.3 grams of proteasome inhibitor in a mixture containing 3.0 grams mineral oil/96.2 grams white petrolatum by stirring in a 100 ml beaker while heating sufficiently to dissolve both compounds. The mixture is sterile filtered through a 0.22 μm filter at a sufficient temperature to be filtered and filled aseptically into sterile ophthalmic ointment tubes.

### Example 5

Hydroxypropylmethyl cellulose (HPMC), sodium chloride, EDTA sodium, and surfactant are dissolved in a beaker containing approximately ⅓ of the final weight of water and stirred for 10 minutes with an overhead stirrer. The mixture is sterilized by autoclaving at 121° C., for 20 minutes. A proteasome inhibitor and a steroid are dry heat sterilized and added to the HPMC-containing solution by aseptic powder addition. Mannitol, Poloxamer 407, BAK, and boric acid are dissolved separately in approximately ½ of the final weight of water and added by sterile filtration (0.22 um filter) and stirred for 10 minutes to form a mixture. The mixture is adjusted to the desired pH with sterile sodium hydroxide (1N to 10N) while stirring, brought to a final weight with sterile water, and aseptically dispensed into multi-dose containers.

### Example 6

Noveon AA-1 is slowly dispersed into a beaker containing approximately ⅓ of the final weight of water and stirred for 1.5 hrs., with an overhead stirrer. EDTA, sodium chloride, and surfactant are then added to the polymer solution and stirred for 10 minutes after each addition. The polymer suspension is at a pH of about 3.0-3.5. The mixture is sterilized by autoclaving at 121° C. for 20 minutes. The proteasome inhibitor and steroid, as indicated in table 2, are dry heat sterilized and added to the polymer suspension by aseptic powder addition. BAK, mannitol, and boric acid are dissolved separately in approximately ½ of the final weight of water, added to the polymer mixture by sterile filtration (0.22 um filter) and stirred for 10 minutes. The mixture is adjusted to the desired pH with sterile sodium hydroxide (1N to 10N) while stirring, brought to a final weight with sterile water, and aseptically dispensed into multi-dose containers.

The present invention is not to be limited in scope by the specific embodiments described herein. Indeed, various modifications of the invention in addition to those described will become apparent to those skilled in the art from the foregoing description. Such modifications are intended to fall within the scope of the appended claims.

Various publications are cited herein, the disclosures of which are incorporated by reference in their entireties.

We claim:

1. A composition for ocular administration, comprising a proteasome inhibitor, a proteasome inhibitor derivative, or a pharmaceutically-acceptable salt thereof, in a carrier for administration to the eye, wherein the proteasome inhibitor has one of the following formulas: 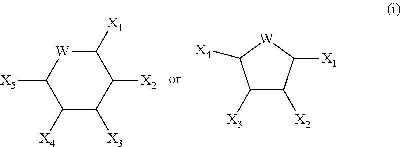
wherein W is selected from the group consisting of a methyl group, an alkyl group, a methylene group, an amine group, an acyl group, a carbonyl group, an oxygen atom, a sulfur atom, and wherein X₁ to X₅ are independently selected from the group consisting of a hydrogen atom, a halogen, a hydroxyl group, an ether group, an alkyl group, an aryl group, a nitro group, a cyano group, a thiol group, a thioether group, an amino group, an amido group, and an OR group, where R is an ester of a dihydrocinnamate; or
(ii) a dihydrocinnamate compound selected from the group consisting of 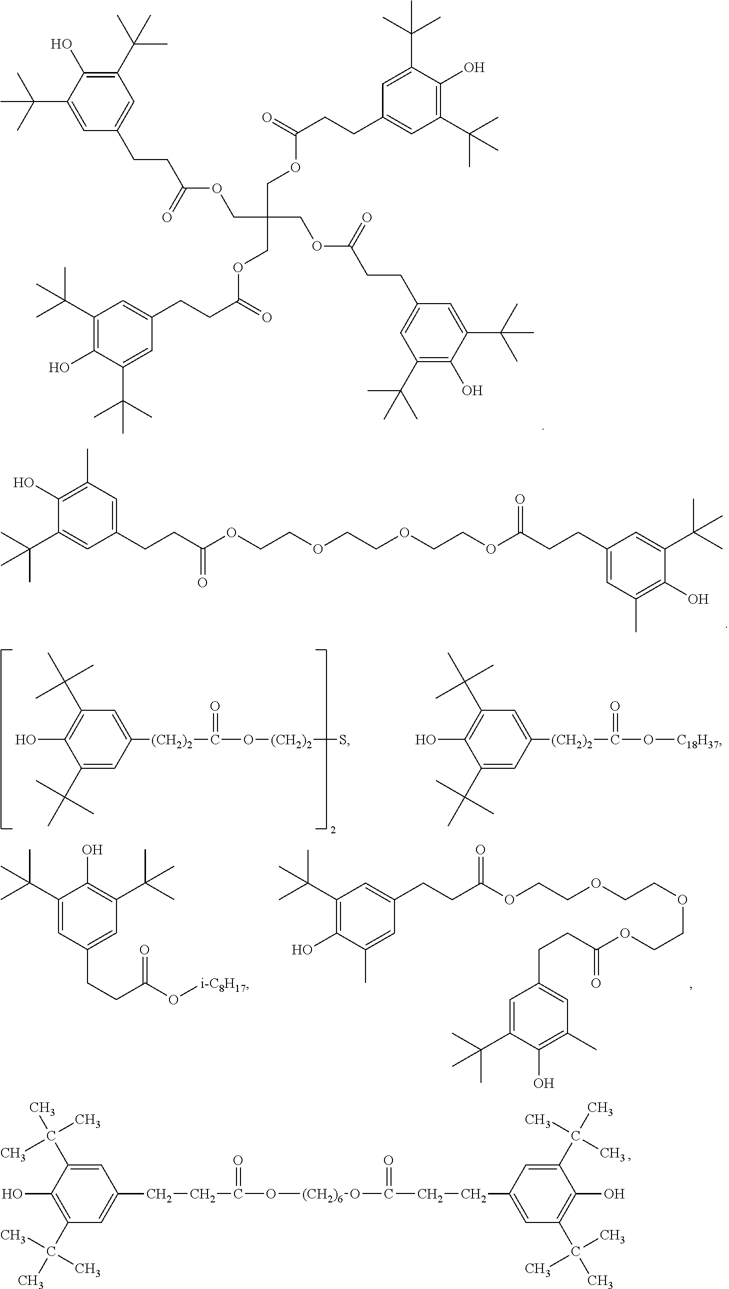 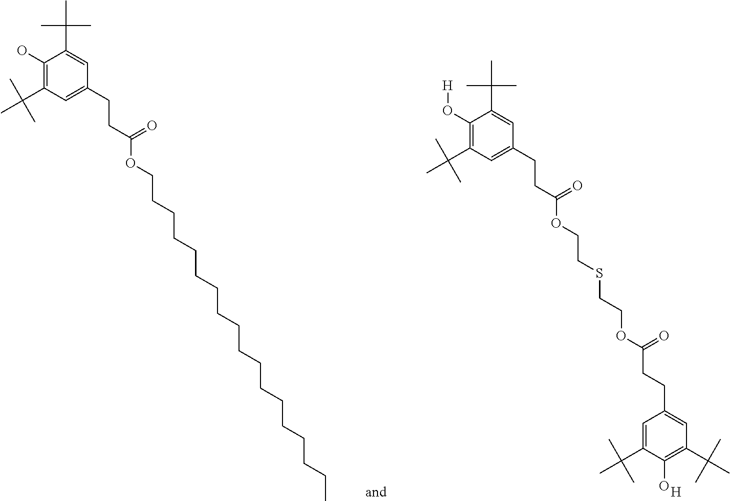
and analogs of the compounds in (i) or (ii) wherein one to three of the hydrogen atoms on the aromatic ring in the dihydrocinnamate moiety is replaced with a moiety selected from the group consisting of halogen, hydroxyl, ether, C₁₋₆ alkyl, C₆₋₁₀ aryl, nitro, cyano, thiol, thioester, amino, and amido.

2. The composition of claim 1, further comprising one or more additional active agents, selected from the group consisting of anti-inflammatory agents, antimicrobial agents, anesthetics, and anti-proliferative agents.

3-4. (canceled)

5. The composition of claim 1, in the form of eye drops or other topical formulations for direct administration to the eye.

6. The composition of claim 1, in the form of an injectable formulation suitable for subconjunctival injections, periocular injections, or intravitreal injections.

7. The composition of claim 1, in the form of an implant.

8. The composition of claim 7, wherein the surgical implant provides sustained release of the proteasome inhibitor.

9. The composition of claim 1, wherein the composition is in the form of an ophthalmic solution comprising water, a polymeric suspending agent, and the proteasome inhibitor, wherein said composition has a pH of about 6.0 to 6.6.

10-11. (canceled)

12. The composition of claim 9, wherein said composition is incorporated into a formulation administrable in a depot format.

13. (canceled)

14. The composition of claim 9, further comprising one or more agents selected from the group consisting of: a buffering agent, an osmolarity adjusting agent, disodium EDTA, a polymeric suspending agent, and a water-swellable water-insoluble crosslinked carboxy-vinyl polymer that comprises at least 90% acrylic acid monomers and about 0.1% to about 5.0% crosslinking agent.

15. The composition of claim 1, in the form of a solid, semi-solid, powdered, or lyophilized composition comprising a polymeric suspending agent, which upon addition of water produces an aqueous formulation having a pH from about 6.0 to about 6.6.

16. (canceled)

17. The composition of claim 15, further comprising one or more agents selected from the group consisting of: a solubilizing agent, a buffering agent, an osmolarity adjusting agent, a chelating agent, disodium EDTA, a polymeric suspending agent, and a water-swellable water-insoluble crosslinked carboxy-vinyl polymer that comprises at least 90% acrylic acid monomers and about 0.1% to about 5.0% crosslinking agent.

18. The composition of claim 15, wherein the proteasome inhibitor is present at a concentration of about 0.1% to about 0.5% by weight.

19. A method for treating ocular disorders associated with proteasome activity, comprising administering to a mammal a composition comprising a pharmaceutically acceptable carrier and a pharmaceutically effective amount of one or more proteasome inhibitors selected from the group consisting of: 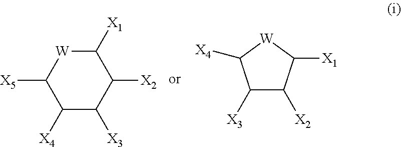
wherein W is selected from the group consisting of a methyl group, an alkyl group, a methylene group, an amine group, an acyl group, a carbonyl group, an oxygen atom, a sulfur atom, and wherein X₁ to X₅ are independently selected from the group consisting of a hydrogen atom, a halogen, a hydroxyl group, an ether group, an alkyl group, an aryl group, a nitro group, a cyano group, a thiol group, a thioether group, an amino group, an amido group, and an OR group, where R is an ester of a dihydrocinnamate; or
(ii) a dihydrocinnamate compound selected from the group consisting of 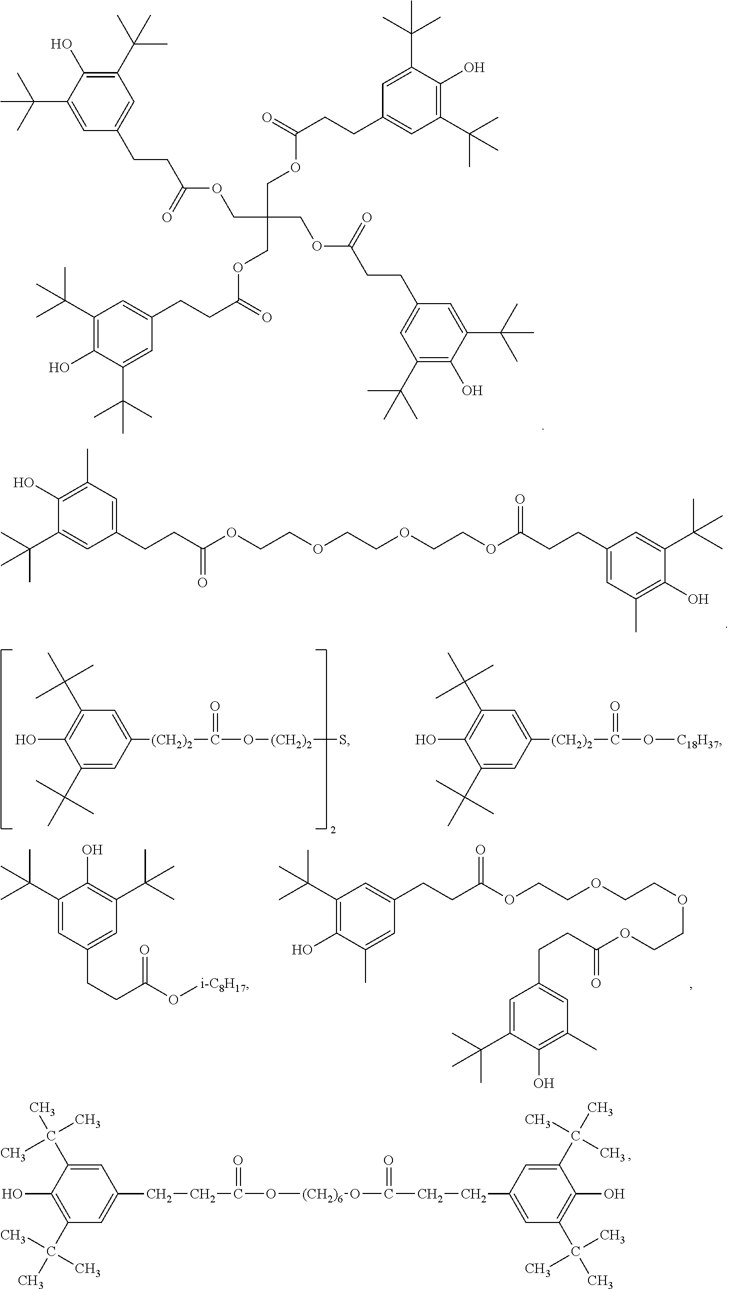 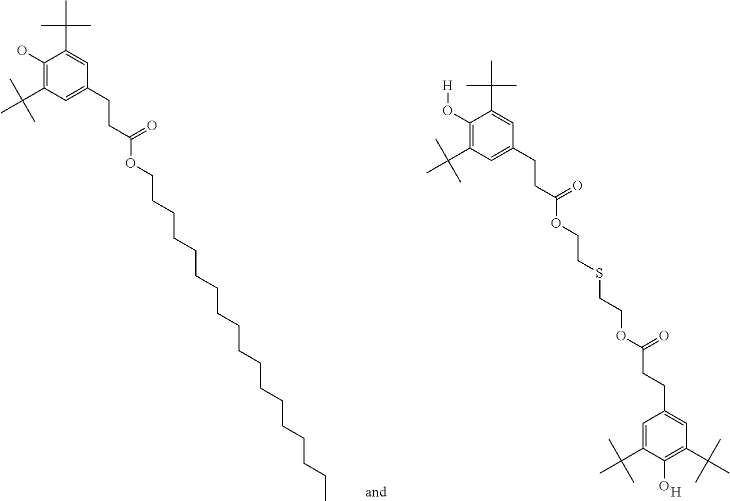
and analogs of the compounds in (i) or (ii) wherein one to three of the hydrogen atoms on the aromatic ring in the dihydrocinnamate moiety is replaced with a moiety selected from the group consisting of halogen, hydroxyl, ether, C₁₋₆ alkyl, C₆₋₁₀ aryl, nitro, cyano, thiol, thioester, amino, and amido.

20. The method of claim 19, wherein the disorder is ocular rosacea.

21. The method of claim 19, wherein the ocular disorder is selected from the group consisting of ocular rosacea, wet and dry age-related macular degeneration (AMD), diabetic retinopathy (DR), glaucoma, neovascular glaucoma, retinal vasculitis, uveitis, keratoconjunctivitis sicca, conjunctivitis, retinitis secondary to glaucoma, neovascular glaucoma, episcleritis, scleritis, optic neuritis, retrobulbar neuritis, ocular inflammation following ocular surgery, ocular inflammation resulting from physical eye trauma, cataract, ocular allergy, dry eye, blepharitis, meibomian gland dysfunction, neurodegenerative disorders affecting the retina, and other retina-specific illnesses with UPS or TNF-alpha involvement.

22. The method of claim 19, wherein the disorder is or results from an ocular bacterial infection.

23. The method of claim 22, wherein bacterial infection is trachoma or bacterial conjunctivitis.

24-27. (canceled)

28. The method of claim 19, wherein the composition further includes one or more additional active agents, selected from the group consisting of anti-inflammatory agents, antimicrobial agents, anesthetics, and anti-proliferative agents.

29. The method of claim 28, wherein the anti-inflammatory agent is a steroid.

30. The method of 19, wherein said composition is topically applied to the eye.

31. The method of claim 19, wherein said composition is injected into the eye.

32. The method of claim 19, wherein said composition is to be administered as a depot, and wherein said composition contains sufficient proteasome inhibitor to provide a sustained release of the administration of the proteasome inhibitor to the target tissue for at least about 12 hours.

33-44. (canceled)
